(19) United States
(12) Patent Application Publication
(10) Pub. No.: US 2023/0173098 A1
(43) Pub. Date: Jun. 8, 2023

(54) NANOPARTICLE COMPLEX FOR ORAL ADMINISTRATION AND USES THEREOF

(71) Applicant: IUCF-HYU (Industry-University Cooperation Foundation Hanyang University), Seoul (KR)

(72) Inventors: DONG YUN LEE, Seongnam-si (KR); Hyung Shik KIM, Seoul (KR)

(21) Appl. No.: 18/073,939

(22) Filed: Dec. 2, 2022

(30) Foreign Application Priority Data
Dec. 6, 2021 (KR) 10-2021-0173100
Jun. 20, 2022 (KR) 10-2022-0074985

(51) Int. Cl. A61K 47/69; A61K 41/00; A61K 47/64; A61K 9/00; A61P 35/00

(52) U.S. Cl. CPC A61K 47/6929; A61K 41/0061; A61K 41/0052; A61K 47/644; A61K 41/0071; A61K 9/0053; A61P 35/00

(57) ABSTRACT

Provided are a nanoparticle complex for oral administration, a pharmaceutical composition for oral administration for treating brain tumors, including the same, a pharmaceutical composition for oral administration for photothermal therapy (PTT) or photodynamic therapy (PDT), and a method of treating brain tumors using the pharmaceutical composition, and the nanoparticle complex for oral administration has not only excellent metal-enhanced fluorescence (MEF) but also excellent metal-enhanced reactive oxygen generation (MERos) due to a surface plasma resonance effect through a bond with the photosensitizer while having excellent oral absorption rate through a bond with lactoferrin, and when a pharmaceutical composition including the nanoparticle complex is administered, the pharmaceutical composition can effectively permeate the small intestinal epithelium and the blood-brain barrier without toxicity and can be effectively accumulated in brain tumor tissue to have an excellent photothermal therapy (PTT) or photodynamic therapy (PDT) effect in brain tumor tissue. In addition, there is an advantage in that brain tumors can be effectively treated by adjusting the order of photothermal therapy (PTT) and photodynamic therapy (PDT), and the like after administering a pharmaceutical composition including a nanoparticle complex for oral administration.

### CROSS-REFERENCE TO RELATED APPLICATION

This application claims priority to and the benefit of Korean Patent Application No. 10-2021-0173100, filed on Dec. 6, 2021, and Korean Patent Application No. 10-2022-0074985, filed on Jun. 20, 2022, the disclosures of which are incorporated herein by reference in its entirety.

### BACKGROUND

### 1. Field of the Invention

The present invention relates to a nanoparticle complex for oral administration, a pharmaceutical composition for oral administration for treating brain tumors, including the same, a pharmaceutical composition for oral administration for photothermal therapy (PTT) or photodynamic therapy (PDT), and a method of treating brain tumors using the pharmaceutical composition.

### 2. Discussion of Related Art

Korea has the fastest aging population in the world, and the demand for drugs for degenerative brain diseases and geriatric diseases in this regard is rapidly increasing. In particular, cancer is the leading cause of death among people aged 65 and over, accounting for 865.4 deaths per 100,000 people, and cerebrovascular disease (410.7), heart disease (332.6), and diabetes (146.6) follow. In particular, according to data published by the Korea Central Cancer Registry in 2013, there were 1,679 cases of brain tumors for both men and women, and by age group, those in their 50s accounted for the most with 17.6%, those in their 60s accounted for 16.5% and followed by those in their 70s with 14.9%, showing a high incidence rate in the older age groups. Frequent administration of intravenous injections with a short half-life to elderly patients may not only induce stress in the patient, but may also make it impossible to expect an effective therapeutic effect. Accordingly, there is an urgent need for developing a brain tumor therapeutic agent for oral absorption, which can provide convenience to patients through oral administration with a long half-life and can prevent stress induced by drug administration.

Meanwhile, there are three major methods for treating brain tumors, the first being a surgical operation, the second being radiation therapy, and the third being chemotherapy, and the like. Currently, drugs such as Avastin, temozolomide, and doxorubicin, which are anticancer agents used in clinical practice, are widely used to treat brain tumors, but they are known to have severe side effects such as nausea and vomiting, stomatitis, diarrhea, abdominal pain, alopecia, and infections (pneumonia and urinary tract infection). As other therapeutic methods, gene therapy, immunotherapy, photothermal therapy (PTT), photodynamic therapy (PDT), and the like are currently being studied.

However, nanoparticles used in photothermal therapy (PTT) have a technical problem in reaching brain tumor tissue with high efficiency through the blood-brain barrier (BBB). Further, photosensitizers used in photodynamic therapy (PDT) have a problem in that they have difficulties in reaching and accumulating in brain tumor tissue with high efficiency due to their low water solubility. In addition, since the nanoparticles or photosensitizers have a disadvantage of having a very low oral absorption rate due to the low pH in the gastrointestinal tract even in therapeutic methods including gold nanoparticles, there is a limitation in utilizing them as therapeutic agents.

Therefore, there is a need for developing a complex capable of being orally administered while including gold nanoparticles suitable for photothermal therapy (PTT) and photodynamic therapy (PDT).

### SUMMARY OF THE INVENTION

Objects for solving the aforementioned problems are as follows.

An object is to provide a nanoparticle complex for oral administration, in which gold nanoparticles coated with glutathione; a photosensitizer bonded to the gold nanoparticles; and lactoferrin bonded to the gold nanoparticles are bonded.

Further, another object is to provide a pharmaceutical composition for oral administration or a pharmaceutical composition for oral administration for photothermal therapy (PTT) or photodynamic therapy (PDT), including the nanoparticle complex for oral administration.

In addition, another object is to provide a method of treating brain tumors using the pharmaceutical composition for oral administration.

The nanoparticle complex for oral administration according to an aspect of the present invention includes: gold nanoparticles coated with glutathione; a photosensitizer bonded to the gold nanoparticles; and lactoferrin bonded to the gold nanoparticles.

The photosensitizer may have a substituted thiourea group to be disulfide-bonded to gold nanoparticles.

The photosensitizer may be one or more selected from the group consisting of a chlorin-based compound, a porphyrin-based compound, a bacteriochlorin-based compound, a phthalocyanine-based compound, a naphthalocyanine-based compound, and a 5-aminoevuline ester-based compound.

The lactoferrin may be surface-modified with a biocompatible polymer.

The biocompatible polymer may be one or more selected from the group consisting of polyethylene glycol, polycaprolactone, polylactic acid, polyglycolic acid, polylactate-co-glycolic acid, poly(3-hydroxybutyrate-co-3-hydroxyvalerate) (PHBV), poly(L-lactide-co-caprolactone), and poly(L-lactide-co-D-lactide).

The biocompatible polymer may be substituted with a thiol group (—SH) to be disulfide-bonded to gold nanoparticles.

The nanoparticle complex for oral administration may have an average diameter of 5 nm to 20 nm.

The weight ratio of the gold nanoparticles:the photosensitizer:the lactoferrin may be 19.7 to 20.0:90.0 to 88.0:1.

The pharmaceutical composition according to another aspect of the present invention is characterized by including the nanoparticle complex for oral administration, an isomer thereof, or a pharmaceutically acceptable salt thereof.

The pharmaceutical composition for oral administration may be for treating brain tumors.

The pharmaceutical composition for oral administration for photothermal therapy (PTT) or photodynamic therapy (PDT) according to still another aspect of the present invention is characterized by including the nanoparticle complex for oral administration.

The pharmaceutical composition for oral administration for photothermal therapy (PTT) or photodynamic therapy (PDT) may be targeted to brain tumor tissue.

The method of treating brain tumors according to yet another aspect of the present invention is characterized by including: orally administering the pharmaceutical composition for photothermal therapy (PTT) or photodynamic therapy (PDT); forming a region to be treated by accumulating the pharmaceutical composition in brain tumor tissue; irradiating the region to be treated with output light for photodynamic therapy (PDT); and irradiating the region to be treated with output light for photothermal therapy (PTT).

The nanoparticle complex for oral administration of the present invention has not only excellent metal enhanced fluorescence (MFF), but also metal-enhanced reactive oxygen generation (MERos) due to a surface plasmon resonance effect by being bonded to a photosensitizer while having an excellent oral absorption rate by being bonded to lactoferrin.

Further, a pharmaceutical composition including a nanoparticle complex for oral administration can effectively permeate the small intestinal epithelium and the blood-brain barrier without toxicity and can be effectively accumulated in brain tumor tissue to have an excellent photothermal therapy (PTT) or photodynamic therapy (PDT) effect in brain tumor tissue.

In addition, there is an advantage in that brain tumors can be effectively treated by adjusting the order of photothermal therapy (PTT) and photodynamic therapy (PDT), and the like after administering a pharmaceutical composition including a nanoparticle complex for oral administration.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a schematic view illustrating a procedure in which metal enhanced fluorescence (MEF) and metal-enhanced reactive oxygen generation (MERos) occur in a nanoparticle complex for oral administration according to an embodiment;

FIG. 2 is a view schematically illustrating a procedure of preparing a nanoparticle complex for oral administration (Ce6-AuNPs-Lf) to which lactoferrin and a photosensitizer are bonded;

FIG. 3 illustrates FT-IR spectra for confirming thiourea binding to a photosensitizer (Ce6) through amide formation according to Example 1-1;

FIG. 4 illustrates UV-vis spectra of a photosensitizer (Ce6), gold nanoparticles (AuNP), and a nanoparticle complex for oral administration (Ce6-AuNP-Lf);

FIG. 5 is a graph (data is expressed in mean±SEM; n=3) showing the surface charge of a photosensitizer (Ce6), gold nanoparticles to which a photosensitizer is bonded (Ce6-AuNP), and a nanoparticle complex for oral administration (Ce6-AuNP-Lf);

FIG. 6 illustrates the resulting TEM images and energy-dispersive X-ray spectroscopy (EDX) images of gold nanoparticles to which a photosensitizer is bonded (Ce6-AuNP) and a nanoparticle complex for oral administration (Ce6-AuNP-Lf) (enlarged image: yellow square box of the original image. scale bar: 50 nm);

FIG. 7 is a graph showing the hydrodynamic size percentages of gold nanoparticles to which a photosensitizer is bonded (Ce6-AuNP) and a nanoparticle complex for oral administration (Ce6-AuNP-Lf);

FIG. 8 is a graph showing temperature changes according to PTT laser irradiation time for gold nanoparticles to which a photosensitizer is bonded (Ce6-AuNP) and a nanoparticle complex for oral administration (Ce6-AuNP-Lf);

FIG. 9 is a set of graphs showing the hydrophobic assay of a simple photosensitizer (Ce6), gold nanoparticles to which a photosensitizer is bonded (Ce6-AuNP) and a nanoparticle complex for oral administration (Ce6-AuNP-Lf);

FIG. 10 is a graph showing the Ce6 photo-bleaching rate of a simple photosensitizer (free-Ce6), gold nanoparticles to which a photosensitizer is bonded (Ce6-AuNP) and a nanoparticle complex for oral administration (Ce6-AuNP-Lf) by PDT irradiation;

FIG. 11 illustrates the fluorescence excitation spectra of a simple photosensitizer (free-Ce6) and gold nanoparticles to which a photosensitizer is bonded (Ce6-AuNP);

FIG. 12 is a graph showing the SOSG analysis results of a simple photosensitizer (free-Ce6), gold nanoparticles to which a photosensitizer is bonded (Ce6-AuNP) and a nanoparticle complex for oral administration (Ce6-AuNP-Lf);

FIG. 13A is a graph showing the SOSG analysis results of gold nanoparticles to which a photosensitizer is bonded (Ce6-AuNP) and a nanoparticle complex for oral administration (Ce6-AuNP-Lf) according to the application order of PDT and PTT;

FIG. 13B is a schematic view illustrating a mechanism that may occur when PTT+PDT (application of PDT after application of PTT) is applied to the nanoparticle complex for oral administration;

FIGS. 14A to 14C are UV-vis absorbance graphs of Ce6-AuNP-Lf under conditions mimicking the gastrointestinal microenvironment to which a material is exposed when orally administered (FIG. 14A, pH 2 for 24 hours; FIG. 14B, pH 5 for 24 hours; and FIG. 14C, pH 2 for 3 hours and changed to pH 5 for an additional 12 hours);

FIGS. 15A and 15B are UV-vis absorbance graphs of Ce6-AuNP-Lf in PBS (FIG. 15A) and 10% FBS (FIG. 15B) for 180 days;

FIG. 16 illustrates TEM images showing the morphology of human vascular endothelial cells (HUVECs) and small intestine endothelial cells (Caco-2) treated with AuNP, Ce6-AuNP, and Lf-PEG-AuNP;

FIGS. 17A and 17B are graphs showing the cell viability of small intestine endothelial cells (Caco-2) (FIG. 17A) and human vascular endothelial cells (HUVECs) (FIG. 17B) treated with AuNP, Ce6-AuNP, and Lf-PEG-AuNP for 24 hours;

FIG. 18 is a graph showing TEER values obtained through a cell monolayer after treating small intestine endothelial cells (Caco-2) with free-Lf, Ce6, Ce6-AuNP, Ce6-AuNP-Lf, and pretreated-Lf/Ce6-AuNP-Lf for 24 hours;

FIG. 19 illustrates monolayer TEM images of small intestine endothelial cells (Caco-2) treated with Ce6-AuNP and Ce6-AuNP-Lf;

FIG. 20 is a schematic view of a pretreated-Lf/Ce6-AuNP-Lf group for confirming only passive transport of Ce6-AuNP-Lf in a Caco-2 cell monolayer;

FIG. 21 is a schematic view of a human BBB Transwell model;

FIG. 22 is a graph showing the apparent permeability (Papp) of a control (Con), Ce6-AuNP, Ce6-AuNP-Lf, and pretreated-Lf/Ce6-AuNP-Lf using FITC-dextran;

FIG. 23 is a graph showing the absolute amount of gold nanoparticles permeating the BBB model of a control (Con), Ce6-AuNP, Ce6-AuNP-Lf, and pretreated-Lf/Ce6-AuNP-Lf as measured by ICP-MS;

FIG. 24 illustrates TEM images of the BBB model treated with a control (Con), Ce6-AuNP, Ce6-AuNP-Lf, pretreated-Lf/Ce6-AuNP-Lf, and Clathrin inhibitor/Ce6-AuNP-Lf;

FIG. 25 illustrates confocal images after treating brain tumor cells (U87MG) with Ce6, Ce6-AuNP, and Ce6-AuNP-Lf, respectively;

FIG. 26 is a set of graphs of Annexin V-DY-634/PI apoptosis staining (ab214484, UK) for detecting apoptosis generated by irradiating brain tumor (U87MG) cells with a laser;

FIG. 27 is a graph showing cell viability after irradiating brain tumor (U87MG) cells with a laser;

FIG. 28 is a set of images illustrating DCFH-DA fluorescence signals after irradiating brain tumor (U87MG) cells with a laser (excitation/emission at 485 nm/535 nm);

FIG. 29 is a graph showing the ROS production rate versus DCFH-DA fluorescence signals after irradiating brain tumor (U87MG) cells with a laser;

FIG. 30 is a graph showing the pharmacokinetic results of administering Ce6-AuNP-Lf to Balb/c mice at 30 mg/kg, 60 mg/kg and 5 mg/kg via subcutaneous (SC), oral, and intravenous (IV) administration, respectively;

FIG. 31 illustrates a set of fluorescence tracer images of orally administered Ce6-AuNP-Lf;

FIG. 32 is a graph showing AuNP accumulation rates in normal brain and GBM brain rat models 24 hours after intravenous (IV) and oral administration of Ce6-AuNP-Lf and Ce6-AuNP;

FIG. 33 is a set of bio-TEM images of GBM brain and normal brain after orally administering 60 mg/kg Ce6-AuNP-Lf;

FIG. 34 is a graph confirming specific GBM targeting of Ce6-AuNP-Lf through thermal conversion characteristics under PTT laser after oral administration (60 mg/kg) for 24 hours;

FIG. 35 is a graph showing the ROS production results of Ce6-AuNP and Ce6-AuNP-Lf in 1 cm thick live mouse skin tissue irradiated with a PDT laser (671 nm);

FIG. 36 is a schematic view of a treatment cycle including drug administration, laser irradiation, survival rate and body weight change monitoring in an orthotopic GBM mouse model;

FIG. 37 is a graph showing the body weight conversion of an orthotopic GBM mouse model;

FIG. 38 is a graph showing the survival rate after administration of Ce6-AuNP-Lf up to 44 days in an orthotopic GBM mouse model;

FIG. 39 is a graph showing the percentage of GBM in an orthotopic GBM mouse model;

FIG. 40 illustrates a set of images showing the results of Nissl staining, H&E staining, and TUNEL staining of the brain of an orthotopic GBM mouse model;

FIG. 41 is a schematic view including drug administration, laser irradiation, and blood collection for cytokine analysis in an orthotopic GBM (U87MG) rat model;

FIG. 42 is a set of graphs showing the ELISA results of pro-inflammatory cytokines IL-6, TNF-α, and TNF-7 in blood collected after Treatment #1, Treatment #2, and Treatment #3 according to FIG. 41;

FIG. 43 is a graph showing the percentage of GBM in an orthotopic GBM rat model;

FIG. 44 illustrates a set of images showing the results of Nissl staining and H&E staining of the brain of an orthotopic GBM rat model;

FIG. 45 illustrates a set of images showing immunofluorescent staining of Ki67 and HMGB1 in GBM tissue from an orthotopic GBM rat model;

FIG. 46 is a graph quantifying the mean fluorescence intensity (MFI) of Ki67 in GBM tissue from an orthotopic GBM rat model; and

FIG. 47 is a graph quantifying the mean fluorescence intensity (MFI) of HMGB1 in GBM tissue from an orthotopic GBM rat model.

### DETAILED DESCRIPTION OF EXEMPLARY EMBODIMENTS

The above objects, other objects, characteristics, and advantages will be easily understood through the following preferred embodiments related to the accompanying drawings. However, the present invention is not limited to the embodiments described herein, and may be implemented in various different forms. Rather, the embodiments introduced herein are provided to make the disclosed content thorough and complete, and sufficiently convey the technical spirit to those skilled in the art.

In the description of each drawing, like reference numerals are used for like constituent elements. In the accompanying drawings, the dimensions of the structures are illustrated while being enlarged compared with actual dimensions for clarity of the present invention. Terms such as first and second may be used to describe various constituent elements, but the constituent elements are not limited by the terms. The terms are used only to distinguish one constituent element from another constituent element. For example, without departing from the scope of the invention, a first constituent element may be called a second constituent element, and similarly, the second constituent element may be called the first constituent element. Singular expressions include plural expressions unless the context clearly indicates otherwise.

In the present specification, it will be appreciated that the term “include” or “have” is intended to designate the presence of characteristics, numbers, steps, operations, constituent elements, and parts described in the specification or combinations thereof, and does not exclude in advance the possibility of the presence or addition of one or more other characteristics, numbers, steps, operations, constituent elements, and components, or combinations thereof. Furthermore, a case where a part such as a layer, a film, a region, and a plate is present “on” another part includes not only a case where the part is present “directly on” another part, but also a case where still another part is present therebetween. Conversely, a case where a part such as a layer, a film, a region, and a plate is present “under” another part includes not only a case where the part is present “directly below” another part, but also a case where still another part is present therebetween.

Unless otherwise specifically described, all numbers, values, and/or expressions for expressing quantities of ingredients, reaction conditions, polymer compositions, and mixtures, which are used in the specification, are to be understood as modified in all instances by the term “about” because these numbers are essentially approximations that are reflective of, among other things, various uncertainties of measurement encountered in obtaining such values. In addition, when a numerical range is disclosed in the present description, the numerical range is continuous, and includes, unless otherwise indicated, every value from a minimum value to a maximum value of the numerical range. Furthermore, when the numerical range refers to integers, unless otherwise indicated, the integers include every integer from a minimum value to a maximum value of the numerical range.

It will be appreciated that throughout the present specification, when a range is described for a variable, the variable includes all the values in the described range including the end points described in the range. It will be appreciated that for example, a range of “5 to 10” includes not only values of 5, 6, 7, 8, 9, and 10, but also any sub-range of 6 to 10, 7 to 10, 6 to 9, 7 to 9, and the like, and also includes any value between appropriate integers within the described range, such as 5.5, 6.5, 7.5, 5.5 to 8.5, and 6.5 to 9. Further, it will be appreciated that for example, a range of “10% to 30%” includes not only all the integers including values of 10%, 11%, 12%, 13%, and the like and up to 30%, but also any sub-range of 10% to 15%, 12% to 18%, 20% to 30%, and the like, and also includes any value between appropriate integers within the described range, such as 10.5%, 15.5%, and 25.5%.

Nanoparticles used in the photothermal therapy (PTT) in the related art have a technical problem in reaching brain tumor tissue with high efficiency through the blood-brain barrier (BBB), and photosensitizers used in the phototherapy (PDT) have a problem in that they have difficulties in reaching and accumulating in brain tumor tissue with high efficiency due to their low water solubility. In addition, since the nanoparticles or photosensitizers have a disadvantage of having a very low oral absorption rate due to the low pH in the gastrointestinal tract even in therapeutic methods including gold nanoparticles, there is a limitation in utilizing them as therapeutic agents.

Accordingly, as a result of intensive studies to solve the above problems, the present inventors found that a nanoparticle complex, in which a photosensitizer and lactoferrin are disulfide-bonded to the surface of glutathione-coated gold nanoparticles has not only excellent metal-enhanced fluorescence (MEF) but also excellent metal-enhanced reactive oxygen generation (MERos) due to a surface plasma resonance effect through a bond with the photosensitizer while having excellent oral absorption rate through a bond with lactoferrin, and found that when a pharmaceutical composition including the nanoparticle complex is administered, the pharmaceutical composition can effectively permeate the small intestinal epithelium and the blood-brain barrier without toxicity and can be effectively accumulated in brain tumor tissue to have an excellent photothermal therapy (PTT) or photodynamic therapy (PDT) effect in brain tumor tissue, thereby completing the present invention.

The nanoparticle complex for oral administration according to an exemplary embodiment is characterized by including: gold nanoparticles coated with glutathione; a photosensitizer bonded to the gold nanoparticles; and lactoferrin bonded to the gold nanoparticles.

In the present invention, gold (Au) nanoparticles are configured to generate heat by surface plasmon resonance upon irradiation with electromagnetic waves, and serve to form the core of the nanoparticle complex. Although iron oxide nanoparticles, silver nanoparticles, or silica nanoparticles may be applied instead of the gold nanoparticles, if necessary, gold nanoparticles having absorbance in the near-infrared region are preferred.

Since metal nanoparticles in the related art have a very low oral absorption rate due to low pH in the gastrointestinal tract, making it difficult for the metal nanoparticles to be utilized as a therapeutic agent, the metal nanoparticles may be bonded to or coated with glutathione such that the biocompatibility of the metal particles may be improved and the stability thereof may be maintained even at low pH. According to an embodiment, the glutathione may form a disulfide bond with the surface of metal nanoparticles to coat the surface of the metal nanoparticles.

According to an embodiment, the mass ratio of the gold nanoparticles:the glutathione may be 1:1 to 3. When the content of glutathione is too low and outside the above range, there is a disadvantage in that it is difficult to maintain the stability of the nanoparticles due to the antioxidant effect of glutathione in the oral absorption route under acidic conditions, and when the content of glutathione is too high and outside the above range, there is a disadvantage in that the treatment of tumors may be adversely affected by enhancing the activity of the antioxidant 7-glutamylcysteine synthetase which suppresses tumor cell apoptosis after tumor cell endocytosis.

In the present invention, the photosensitizer may be bonded to the gold nanoparticles and irradiated with light of a specific wavelength to be energetically excited, and in this case, may cause apoptosis or necrosis of surrounding tumor cells while generating fluorescence signals or producing reactive oxygen species (such as singlet oxygen, oxygen radicals, superoxide and peroxide) by transferring the excited energy to the surrounding substrate or oxygen.

In an exemplary embodiment, the photosensitizer may include one or more selected from the group consisting of a phorphyrin-based compound, a chlorin-based compound, a bacteriochlorin-based compound, a phthalocyanine-based compound, a naphthalocyanine-based compound, and a 5-aminoevuline ester-based compound.

According to an embodiment, chlorin e6 (Ce6) may be used as the photosensitizer. The chlorin e6 may be bonded to gold nanoparticles, preferably, glutathione on the surface of gold nanoparticles.

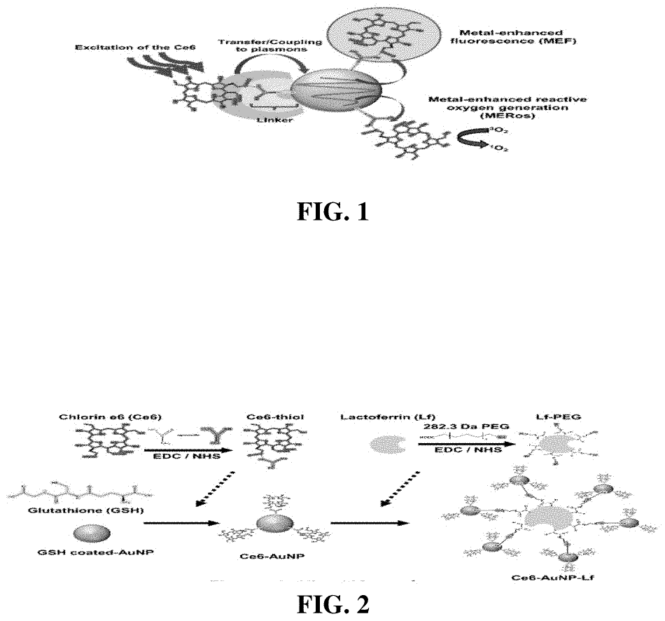

FIG. 1 is a schematic view illustrating a procedure in which metal enhanced fluorescence (MEF) and metal-enhanced reactive oxygen generation (MERos) occur in a nanoparticle complex for oral administration according to an embodiment. Referring to the drawing, not only metal enhanced fluorescence (MEF), but also metal-enhanced reactive oxygen generation (MERos) which induces cell apoptosis and tissue destruction may be generated by a surface plasma resonance effect under the photoactivation of a specific wavelength range.

According to an embodiment, the photosensitizer may have a substituted thiourea group to be disulfide-bonded to gold nanoparticles. As the photosensitizer has a substituted thiourea group, there is an advantage in that a highly reactive disulfide bond reaction can be performed without an additional catalyst, and since the thiourea group bonded to the end of the photosensitizer is bonded to gold nanoparticles through a disulfide bond with glutathione on the surface of the gold nanoparticles, there is an advantage in that a highly efficient photodynamic therapy can be achieved by a metal-enhanced reactive oxygen generation (MERos) phenomenon generated from the gold nanoparticles.

According to an embodiment, the mass ratio of the gold nanoparticles:the photosensitizer may be 1:1 to 5. When the content of the photosensitizer is too low and outside the above range, there is a disadvantage in that the MERos effect cannot be sufficiently exhibited, and when the content of the photosensitizer is too high and outside the above range, there is a disadvantage in drug treatment due to an increase in hydrophobic properties.

As used herein, lactoferrin refers to a material that binds to iron in the human body and changes into a strong antioxidant, and exhibits activities such as inhibition of bacterial growth in the body, and is abundant in milk and contained in the highest content in colostrum among types of milk, and since lactoferrin is a ligand that can bind to a low-density lipoprotein receptor-related protein (LRP), which is one of the cell membrane proteins, and many lactoferrin receptors are expressed in the small intestinal epithelium, lactoferrin may be used as a composition for targeting brain tumors and improving the bioavailability, particularly, oral absorption rate, of metal nanoparticles.

In the present invention, lactoferrin may be surface-modified with a biocompatible polymer to minimize modification of lactoferrin in the gastrointestinal tract and blood circulation processes. By such a configuration, the oral absorption rate of the nanoparticle complex may be improved, and ultimately, the targeting to brain tissue may be enhanced. In addition, there is an advantage of being able to prevent an aggregation phenomenon between nanoparticles and ultimately contribute to the enhancement of a photothermal or photodynamic therapeutic effect by maintaining a certain distance between nanoparticle complexes by the repulsive force between biocompatible polymers bonded to lactoferrin.

According to an exemplary embodiment, the biocompatible polymer may be a polysaccharide monomer or polymer which preferably has biocompatible characteristics, and preferably, the polymer may be polyethylene glycol, polycaprolactone, polylactic acid, polyglycolic acid, polylactate-co-glycolic acid, poly(3-hydroxybutyrate-co-3-hydroxyvalerate) (PHBV), poly(L-lactide-co-caprolactone), and poly(L-lactide-co-D-lactide).

According to an embodiment, polyethylene glycol, which is used as the biocompatible polymer, has an advantage in that by preventing the adsorption of proteases present in vivo onto nanoparticles, the degradation of lactoferrin is prevented and the duration of the nanoparticles in the bloodstream is increased.

According to an embodiment, the mass ratio of lactoferrin:polyethylene glycol may be 1:10 to 100, preferably 1:10 to 50. When the content of polyethylene glycol is too low and outside the above range, there is a disadvantage in that it is not possible to prevent in vivo proteases from being adsorbed onto nanoparticles, and when the content of polyethylene glycol is too high and outside the above range, there is a disadvantage in that lactoferrin may interfere with a binding domain capable of binding to a lactoferrin acceptor.

According to an embodiment, the weight ratio of the gold nanoparticles:the photosensitizer:the lactoferrin may be 19.7 to 20.0:90.0 to 88.0:1. The disadvantages of a content outside the above range are as described above.

According to an embodiment, a gold nanoparticle complex was prepared by reacting polyethylene glycol-bound lactoferrin and a photosensitizer with gold nanoparticles coated with glutathione, and it could be confirmed that the nanoparticle complex maintained the inherent physical characteristics of gold nanoparticles, that is, a significant absorbance peak and exothermic effect in the near-infrared wavelength range.

In another embodiment, it could be confirmed that stability was maintained even under strongly acidic conditions and there is an excellent oral absorption rate-enhancing effect due to low cytotoxicity to small intestine endothelial cells and vascular endothelial cells.

According to the present invention, the nanoparticle complex may be used as a nanoparticle complex for oral administration.

In another embodiment, since it could be confirmed that the nanoparticle complex has an excellent brain tumor tissue targeting effect and an excellent photothermal therapy (PTT) or photodynamic therapy (PDT) effect in a brain tumor animal model, it could be seen that the nanoparticle complex may be utilized for the treatment of brain tumors.

In the present invention, the pharmaceutical composition for oral administration may include a nanoparticle complex for oral administration, an isomer thereof, or a pharmaceutically acceptable salt thereof.

In the present invention, the nanoparticle complex may be used for brain tumor treatment, photothermal therapy, and photodynamic therapy, and may be used for oral administration because the nanoparticle complex exhibits high stability in the gastrointestinal tract in terms of administration method.

As used herein, the term “treatment” refers to all actions that ameliorate or beneficially change symptoms caused by brain tumors by administering the pharmaceutical composition according to the present invention.

As used herein, the “individual” refers to a subject in need of treatment for a brain tumor, and more specifically, refers to a mammal such as a human or a non-human primate, a mouse, a dog, a cat, a horse, and a cow.

“Tumors,” which are a disease to be treated in the present invention, refer to a group of diseases characterized by excessive cell proliferation and infiltration into surrounding tissues when the normal apoptotic balance is disrupted, and in consideration of the brain tissue targeting effect of the nanoparticle complex, the tumors may be preferably brain cancer (brain tumors), more specifically glioblastoma multiforme (GBM), but are not limited thereto.

In the present invention, the pharmaceutical composition may further include a pharmaceutically acceptable carrier in addition to an active ingredient. In this case, the pharmaceutically acceptable carrier is one typically used during formulation, and includes lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, but is not limited thereto. Furthermore, the pharmaceutically acceptable carrier may further include a lubricant, a wetting agent, a sweetening agent, a flavoring agent, an emulsifier, a suspending agent, a preservative, and the like in addition to the above components.

In the present invention, the pharmaceutical composition may be administered orally or parenterally (for example, intravenously, subcutaneously, intraperitoneally or applied topically) according to the desired method, and most preferably administered orally. The dose varies depending on the patient's condition and body weight, severity of the disease, drug form, administration route, and duration, but may be suitably selected by those skilled in the art.

In the present invention, the pharmaceutical composition is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including type of diseases of patients, the severity of disease, the activity of drugs, sensitivity to drugs, administration time, administration route, excretion rate, treatment period, and simultaneously used drugs, and other factors well known in the medical field. The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects in consideration of all the aforementioned factors, and this amount may be easily determined by a person skilled in the art.

In the present invention, the pharmaceutical composition for oral administration may be used as a composition for photothermal therapy using the gold nanoparticle property of generating heat when irradiated with electromagnetic waves. The photothermal therapy refers to a treatment method in which light energy is converted into heat energy when a light beam (light) of a certain wavelength is applied, and the heat energy emitted from the light burns cancer cells. Upon irradiation with a laser, heat is generated by the surface plasma resonance (SPR) phenomenon, and is emitted to the surrounding region to affect the external region (for example, tumor cells). As an example, cancer cells may be killed.

In the present invention, the pharmaceutical composition for oral administration may be used as a composition for photodynamic therapy using a photosensitizer and a light beam. The photodynamic therapy may be a treatment method including the procedure of treating a subject in a pathological state with a photosensitizer and irradiating the photosensitizer with a light beam in order to obtain a therapeutic effect by activating the photosensitizer.

In the present invention, the energy amount of the light beam for generating heat energy or activating the photosensitizer may be appropriately selected and used according to the use environment and purpose. For example, a suitable wavelength, power, power density, energy density, application period proportional to the photosensitizer treatment time, and the like may be appropriately selected and adjusted. As the wavelength of the light beam, any wavelength that may be absorbed by the gold nanoparticles or the photosensitizer can be used, and any wavelength capable of producing a desired biological response in the target cells may be included without limitation.

In the present invention, as a light source that produces the light beam, any light source that supplies necessary light energy to generate heat energy or produce a wavelength capable of activating the photosensitizer can be used. Examples of the light source include a laser, a lamp, an optoelectric magnetic device, a diode, a diode-laser, or the like.

In an embodiment, as a result of administering a nanoparticle complex for oral administration to brain tumor cell tissue or a brain tumor animal model, and then applying treatment by varying the order of photodynamic therapy (PDT) or photothermal therapy (PTT), it could be confirmed that upon photothermal therapy (PTT) after photodynamic therapy (PDT), brain tumor cells were relatively efficiently reduced.

In the present invention, the method of treating brain tumors may include: orally administering a pharmaceutical composition for oral administration; forming a region to be treated by accumulating the pharmaceutical composition in brain tumor tissue; irradiating the region to be treated with output light for photodynamic therapy (PDT); and irradiating the region to be treated with output light for photothermal therapy (PTT).

Hereinafter, the present invention will be described in more detail through Examples. However, these Examples are provided only for exemplarily describing the present invention, and the scope of the present invention is not limited by these Examples.

[Materials, etc.]

Materials

Gold (III) chloride trihydrate (HAuCl₄), glutathione (GSH), lactoferrin (human lactoferrin (hLf)), sodium hydroxide (NaOH), a thiourea solution (H₂NCSNH₂), sodium borohydride (NaBH₄), 100-kDa and 10-kDa MWCO dialysis tubing cellulose membranes, 1-ethyl-3-(3-dimethylaminopropyl) carbodiimide (EDC), n-hydroxysuccinimide (NHS), osmium tetroxide (OsO₄), pepsin, a Spurr low-viscosity embedding kit, paraformaldehyde, cresyl violet-acetate, acetic acid, octanol, CHIR-99021, retinoic acid (RA), collagen IV, fibronectin, chlorpromazine hydrochloride and sodium acetate were purchased from Sigma-Aldrich, Mo., USA. A photosensitizer (Chlorin e6 (Ce6, C₃₄H₃₆N₄O₆, MFCD08669566)) was purchased from Frontier Scientific, USA. A Centricon centrifugal filter (MWCO; 3 kDa, UFC9003) was purchased from Millipore, Germany. Bifunctional poly(ethylene glycol) (SH-PEG-COOH) was purchased from Quanta BioDesign, Plain City, USA. JEM-301 HR-TEM grids were purchased from Nanolab Technologies, N.Y., USA. InstantBlue™ was purchased from Expedeon, UK. A BCA protein assay kit was purchased from Pierce Biotechnology, Rockford, Ill., USA. A Transwell insert was purchased from Coming, Inc., Corning, N.Y., USA. Cell cytotoxicity assay EZ-Cytox was purchased from DoGenBio, Seoul, Korea. 3% isoflurane was purchased from HanaPharm, Seoul, Korea. A DeadEnd Fluorometric Terminal deoxynucleotidyl transferase dUTP nick end labeling (TUNEL) System kit was purchased from Promega, USA. Singlet oxygen sensor green (SOSG, S36002), 1×B-27 Supplement, DMEM/F12, Knockout™ Serum Replacement, Non-Essential Amino Acids (100×), β-mercaptoethanol, human Endothelial SFM and a GlutaMAX™ supplement were purchased from Thermo Fisher Scientific, USA. FITC-Dextran 3 kDa was purchased from Invitrogen, USA. An Annexin V-DY-634/PI apoptosis stain (ab214484), anti-Ki67 antibodies (ab15580), anti-HMGB1 antibodies (ab18256), goat anti-rabbit IgG-H&L Alexa Fluor 488 (ab150077) and goat anti rabbit IgG H&L Alexa Fluor 647 (ab150079) were purchased from Abcam, UK. A DAPI mounting kit was purchased from Vector Laboratories, Inc., Burlingame, Calif., USA. ELISAs for IL6 (KET9007), IFN-γ (KET7017) and TNFα (KET9007) were purchased from Abbkine, Wuhan, China.

Experimental Cell Lines and Animals

Experiments were performed using a human umbilical vein endothelial cell line (HUVEC; LONZA, N.J., USA), a human epithelial colorectal cell line (Caco-2; Korean Cell Line Bank, Seoul, Korea), a C6 rat glioma cell line (Rockville, Md., USA), a U87MG human glioblastoma cell line (Korean Cell Line Bank, Seoul, Korea), human brain microvascular endothelial cells (BMVECs) generated from human induced pluripotent stem cells (IMR90-4 iPSCs; WiCell Research Institute, Madison, Wis., USA). HUVECs (passage numbers 4 to 6) were cultured using an endothelial growth medium (EGM-2 bullet kit; LONZA, N.J., USA).

Caco-2, U87MG and C6 cells were cultured using Dulbecco's modified Eagle's medium (DMEM; GenDEPOT, Tex., USA) containing 10% fetal bovine serum (FBS; GenDEPOT, Tex., USA) and 1% penicillin-streptomycin under standard culture conditions at 37° C. and 5% CO₂.

For the culture protocol for BMVECs derived from IMR90-4 iPSCs, reference was made to that described in the methodology section of human blood-brain barrier culture and formation of a cell monolayer.

In vivo experiments were carried out using five- to seven-week-old male Balb/c nude mice, Balb/c mice (Nara-Bio Company, Seoul, Korea), and seven-week-old male Sprague Dawley (SD) rats (DBL, Incheon, Korea).

All animals were housed under specific pathogen-free conditions and maintained under guidelines of the Institutional Animal Care and Use Committee (IACUC: 2020-0081) of Hanyang University.

### PREPARATION EXAMPLES

FIG. 2 is a schematic view schematically illustrating a procedure of preparing a nanoparticle complex for oral administration (Ce6-AuNPs-Lf) to which lactoferrin and a photosensitizer are bonded. Referring to this, the following Preparation Examples 1 to 4 will be specifically described.

### Preparation Example 1. Preparation of Gold Nanoparticles Coated with Glutathione (AuNPs)

A solution of gold (III) chloride trihydrate (HAuCl₄) (11.1 mM), glutathione (GSH) (37.8 mM) and sodium hydroxide (NaOH) (178 mM) was dissolved in methanol/water (1.3:1 v/v, 20 mL).

Next, this solution was diluted to a final Au³⁺ concentration (0.48 mM) by adding methanol (104 mL) and water (294 mL) thereto. In this case, Au³⁺ was reduced by adding sodium borohydride (NaBH₄) (0.25 M, 4 mL) thereto. The reduction of Au was allowed to proceed for 24 hours at 100° C. with constant stirring.

The gold nanoparticles (AuNPs) produced by the reduction were precipitated by adding NaCl (68 mM) to methanol (200 mL), and then gold nanoparticles (AuNPs) coated with GSH could be finally obtained by centrifugation (3200 rpm, 5 min).

The GSH-coated gold nanoparticles (AuNPs) obtained by the centrifugation were reconstituted in distilled water (DW).

### Preparation Example 2. Preparation of Photosensitizer with Substituted Thiourea

After a photosensitizer (Chlorin e6; Ce6) was diluted with a sodium hydroxide (0.1 M) solution according to the manufacturer's instructions, methanol was added thereto to a final concentration of Ce6 (5 mM), and a photosensitizer solution (Ce6 solution) was prepared by adjusting the pH to 6.2.

Next, the Ce6 solution (108 μL) was mixed with Sulfo-NHS (990 μL, 40.7 mg/mL) and EDC (900 μL, 16 mg/mL) in PBS (10 mM, pH 6.2), and the mixed solution was mixed every 5 minutes for 30 minutes.

Next, a thiourea solution (3006 μL, 4 mM) was added and the solution was occasionally stirred for 120 minutes. Next, sodium hydroxide (42 μL, 0.1 M) was added to quench the reaction.

Next, a Ce6-thiol solution was prepared by removing excess unreacted reagents using a thiourea-conjugated Ce6 (Ce6-thiol) purification process.

Next, after hydrochloric acid (3.3 μL) was added to the Ce6-thiol solution (1 mL), the resulting solution was homogenized and centrifuged at 15,000 rpm for 2 minutes, and a photosensitizer (Ce6-thiol) with a substituted thiourea group could be finally obtained by discarding the supernatant.

In this case, a pellet including the photosensitizer (Ce6-thiol) with the substituted thiourea group was resuspended in DW (600 μL), and this process was repeated twice. Additionally, the pellet was resuspended once more in DW (200 L), and then stored at 4° C.

### Preparation Example 3. Preparation of Nanoparticle Complex (Ce6-AuNPs) Bonded to Photosensitizer

The GSH-coated gold nanoparticles (AuNPs) prepared according to Preparation Example 1 and the thiourea group-substituted photosensitizer (Ce6-thiol) solution (23 μM) prepared according to Preparation Example 2 were mixed at a feed molar ratio of 1:1, allowed to react and stirred for 24 hours.

In this case, the molar extinction coefficients of Ce6 and AuNP were 45,000 cm⁻¹/M and 55,000 cm⁻¹/M at 532 nm and 671.0 nm, respectively.

Molar concentrations of Ce6 and AuNP were calculated by the following Equation 1 by UV-vis spectroscopy according to the Beer-Lambert law.

$$A ( λ ) = {log}_{10} \frac{{I}_{0}}{I} = ε c \qquad [ Equation 1 ]$$

* A (λ) is the measured wavelength-dependent absorbance, I₀ is the incident light intensity, I is the transmitted light intensity, ε is the wavelength-dependent extinction coefficient of the substrate, c is the substrate concentration, and l is the path length (0.1 cm) of the quartz cuvette

After stirring for 24 hours, the unconjugated-Ce6 was removed using a Centricon centrifugal filter having a membrane pore size of 3 kDa NMWCO, and the final product photosensitizer-bonded nanoparticle complex (Ce6-AuNPs) was reconstituted with DW (1 mL).

### Preparation Example 4. Preparation of Nanoparticle Complex (Ce6-AuNPs-Lf) for Oral Administration, Bonded to Lactoferrin and Photosensitizer

Lactoferrin was surface-modified with a biocompatible polymer as follows.

Specifically, lactoferrin-conjugated poly(ethylene glycol) was synthesized by EDC/NHS amide bond conjugation. That is, a PEG mixed solution was prepared by dissolving EDC (250 mM) and NHS (500 mM) in PBS containing PEG (12.5 mM) with constant stirring. After 15 minutes, lactoferrin surface-modified with a biocompatible polymer polyethylene glycol (Lf-PEG) was obtained by adding the PEG mixed solution (12.5 mM) to a PEG mixed solution containing lactoferrin (0.125 mM) at 4° C. with constant stirring for 24 hours.

Next, polyethylene glycol surface-modified lactoferrin (Lf-PEG) was collected by dialysis using a 10-kDa-pore membrane at 4° C. and lyophilized.

Next, a nanoparticle complex (Ce6-AuNPs-Lf) to which lactoferrin and a photosensitizer are bonded was prepared by mixing the solution (70 μM) of the nanoparticle complex (Ce6-AuNPs) bonded to the photosensitizer prepared in Preparation Example 3 with the Lf-PEG solution (5 mL) dissolved in PBS at 4° C. with constant stirring for 24 hours. Next, a nanoparticle complex (Ce6-AuNPs-Lf) bonded to lactoferrin and the photosensitizer was collected by dialysis using a 100-kDa-pore membrane at 4° C. and lyophilized.

### EXAMPLES

### Example 1. Examination of Preparation Process and Physicochemical Properties of Nanoparticle Complex for Oral Administration

1-1. Confirmation of Structure of Nanoparticle Complex for Oral Administration

Through FT-IR spectra, it was intended to identify thiourea binding to the photosensitizer (Ce6) through amide formation

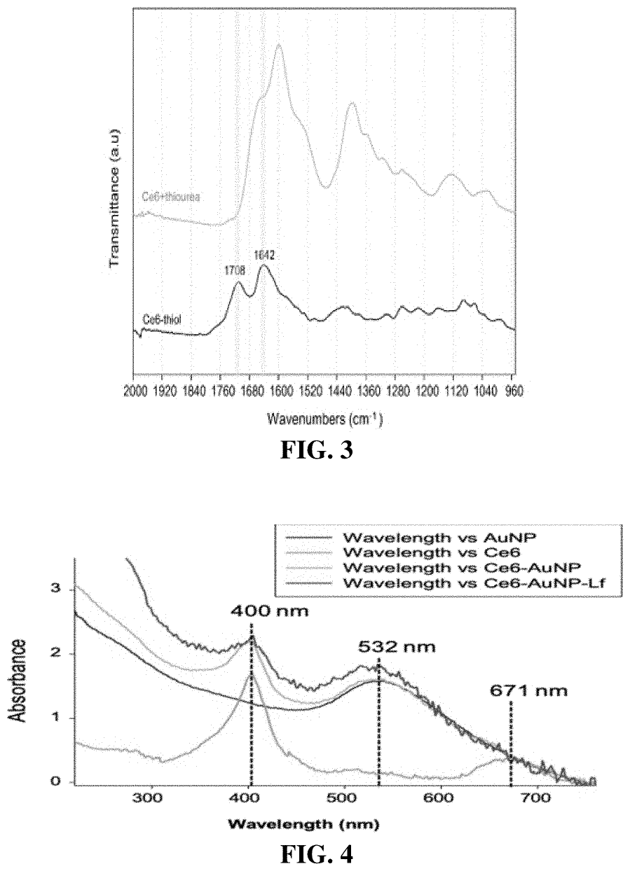

FIG. 3 illustrates FT-IR spectra for confirming thiourea binding to a photosensitizer (Ce6) through amide formation according to Example 1-1.

Specifically, Ce6-thiol is a Ce6 solution modified with thiourea (Example 4 solution), and Ce6+thiourea is a Ce6 solution simply mixed with thiourea (Comparative Example).

Referring to FIG. 3, it could be confirmed that the band at 1708 cm⁻¹ in a range from 1550 cm⁻¹ to 1750 cm⁻¹ corresponds to the C═O stretching of carboxylic acids in Ce6. Further, it could be confirmed that Ce6-thiol has a band at 1642 cm⁻¹ in the vibrational mode of the amide 1 bond. Through this, it could be confirmed that a new radical formed at 1642 cm⁻¹ and a decrease in intensity at 1708 cm⁻¹ indicate modification of carboxylic acid radicals during amide formation.

In addition, observation was made using a UV-visible spectrophotometer (NanoDrop 2000; Thermo Scientific, Wilmington, Del., USA) in order to confirm that the thiourea group-substituted photosensitizer (Ce6-thiol) is bonded to the AuNP surface through a disulfide bond.

FIG. 4 illustrates UV-vis spectra of a photosensitizer (Ce6), gold nanoparticles (AuNP), and a nanoparticle complex for oral administration (Ce6-AuNP-Lf).

Referring to FIG. 4, since it could be confirmed that specific peaks of Ce6 and AuNP were shown at wavelengths of 400 nm/532 nm and 671 nm, it could be confirmed that Ce6-thiol and AuNP were chemically bound by a disulfide bond.

Furthermore, as a result of calculating the binding ratio between Ce6 and AuNP in the Ce6-AuNP using the absorbance measured at each wavelength and the molar extinction coefficients of AuNP and Ce6, it could be confirmed that an average of 4.5 Ce6 were bonded to one AuNP.

1-2. Examination of Physicochemical Properties of Nanoparticle Complex for Oral Administration

Measurements were made using a Zetasizer Nano ZS (Malvern, UK) to confirm the surface charge of a simple photosensitizer, the gold nanoparticles bonded to the photosensitizer (Ce6-AuNP) of Preparation Example 3, and the nanoparticle complex for oral administration (Ce6-AuNP-Lf) of Preparation Example 4, respectively.

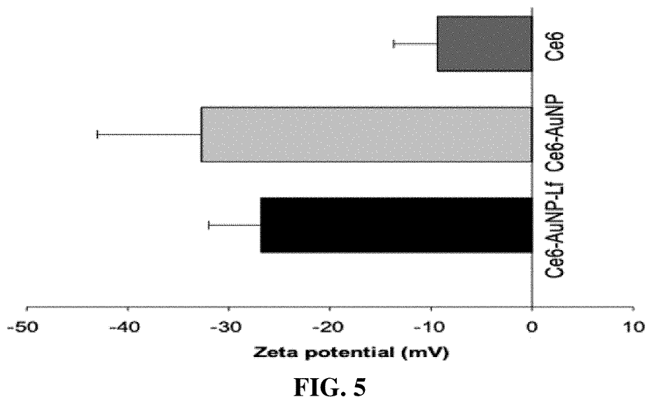

FIG. 5 is a graph (data is expressed in mean±SEM; n=3) showing the surface charge of a photosensitizer (Ce6), gold nanoparticles to which a photosensitizer is bonded (Ce6-AuNP), and a nanoparticle complex for oral administration (Ce6-AuNP-Lf).

Referring to FIG. 5, it could be confirmed that the surface charge of Ce6, Ce6-AuNP and Ce6-AuNP-Lf is −9.4±4.3, −32.7±10.3 and −26.8±5.2, respectively, and through this, it could be confirmed that the surface charge of AuNP is negative due to the carboxyl group of glutathione coated on the gold nanoparticles and Ce6 is also negatively charged due to the carboxyl group functional group, making the surface charge thereof even more negative, thus it could be confirmed that Ce6 is bonded to gold nanoparticles. On the other hand, since lactoferrin is positively charged, the surface charge of Ce6-AuNP-Lf became more positive than that of Ce6-AuNP, and through this, it could be confirmed that the stepwise synthesis proceeded well.

Meanwhile, TEM images were confirmed in order to confirm physical properties such as size, dispersion and aggregation of the gold nanoparticles bonded to the photosensitizer (Ce6-AuNP) of Preparation Example 3 and the nanoparticle complex for oral administration (Ce6-AuNP-Lf) of Preparation Example 4.

In this case, the elemental mapping and sizes of the Ce6-AuNP and Ce6-AuNP-Lf were measured by high-resolution TEM (HR-TEM). Specifically, HR-TEM grids were prepared by placing each sample on a carbon film-covered copper mesh grid for 1 minute, and the grids were allowed to air dry before being imaged by TEM.

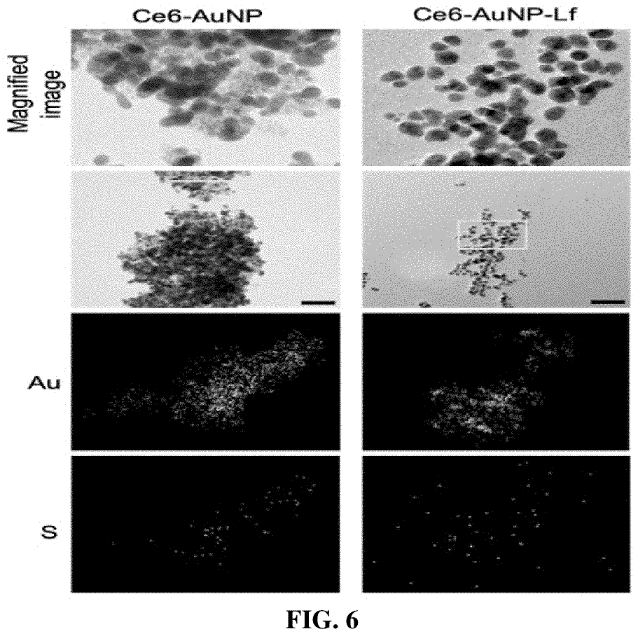

FIG. 6 illustrates the resulting TEM images and energy-dispersive X-ray spectroscopy (EDX) images of gold nanoparticles to which a photosensitizer is bonded (Ce6-AuNP) and a nanoparticle complex for oral administration (Ce6-AuNP-Lf) (enlarged image: yellow square box of the original image. scale bar: 50 nm).

Referring to FIG. 6, it could be confirmed that Ce6-AuNP-Lf was well dispersed, whereas Ce6-AuNP was highly aggregated.

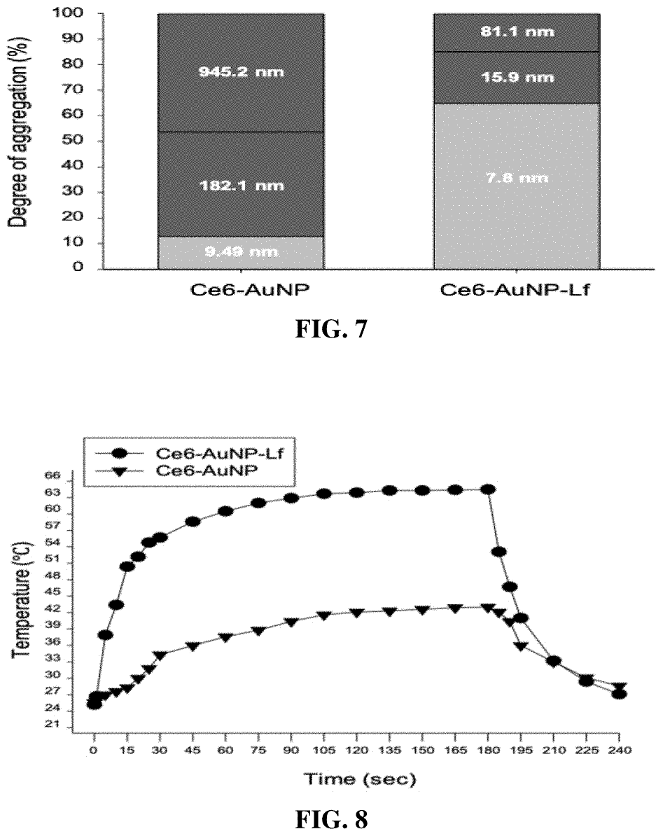

FIG. 7 is a graph showing the hydrodynamic size percentages of gold nanoparticles to which a photosensitizer is bonded (Ce6-AuNP) and a nanoparticle complex for oral administration (Ce6-AuNP-Lf).

Referring to FIG. 7, it could be confirmed that the aggregate diameters of Ce6-AuNPs were actually 945.2±142.4 nm, 182.1±40.8 nm, and 9.49±12.8 nm, confirming hydrodynamic size percentages of 46.4%, 40.8%, and 12.8%, respectively.

Through this, it could be confirmed that cations present in the solution were bonded to the carboxylic acid of glutathione coated on the AuNP surface to neutralize the surface charge, thereby inducing irreversible aggregation of AuNPs into large structures

On the other hand, it could be confirmed that the aggregated diameters of Ce6-AuNP-Lf were found to be within 7.8±2.6 nm, 15.9±6.2 nm, and 81.1±15.4 nm with hydrodynamic size percentages of 64.8%, 20.2%, and 15.1%, respectively.

It could be confirmed that the steric stabilization mediated by PEGylation of Ce6-AuNP-Lf prevented particle aggregation to facilitate dispersion.

In particular, it is desirable to maintain Ce6-AuNP-Lf at an average diameter from 5 nm to 20 nm for the nanoparticle complex for oral administration according to an exemplary embodiment because the surface plasmon resonance (SPR) of AuNP irradiated with PTT laser (532 nm wavelength) is highly dependent on NP size.

Meanwhile, it was intended to evaluate the PTT efficiency and photothermal stability of the nanoparticle complex for oral administration (Ce6-AuNP-Lf).

Specifically, a PTT laser (LRS-0532 DPSS Laser System, 532 nm; laser glow Part Number: R5310B1FL, Toronto, ON, Canada) was applied to vials containing Ce6-AuNP (gold equivalent concentration of 10 μM) or Ce6-AuNP-Lf (gold equivalent concentration of 10 μM) for 240 seconds, the heating profiles of Ce6-AuNP and Ce6-AuNP-Lf were measured using a thermal imaging camera (FLIR C2, Wilsonville, Oreg., USA) and quantified by SigmaPlot 10.0 (System Software, Calif., USA), and the results are shown in FIG. 8 and Table 1.

FIG. 8 is a graph showing temperature changes according to PTT laser irradiation time for gold nanoparticles to which a photosensitizer is bonded (Ce6-AuNP) and a nanoparticle complex for oral administration (Ce6-AuNP-Lf).

TABLE 1

| | Laser | Irradiation | Maximum | PTT |
|---|---|---|---|---|
| | (wavelength, | time | temperature | efficiency |
| | power) | (sec) | (Tₘₐₓ) | (Tₘₐₓ − T₀) |
| AuNP | 532 nm, | 240 sec | 45.4 ± 4.2° C. | 20.3 ± 2.1° C. |
| | 4 W/cm² | | | |
| AuNP-Lf | 532 nm, | 240 sec | 56.3 ± 0.5° C. | 29.6 ± 0.2° C. |
| | 4 W/cm² | | | |
| Ce6-AuNP | 532 nm, | 240 sec | 43.0 ± 3.7° C. | 18.4 ± 1.7° C. |
| | 4 W/cm² | | | |
| Ce6-AuNP- | 532 nm, | 240 sec | 64.5 ± 4.5° C. | 39.3 ± 2.3° C. |
| Lf | 4 W/cm² | | | |

* Tmax=maximum temperature; T₀=initial temperature

Referring to Table 1 and FIG. 8, it could be confirmed that the maximum temperature (Tmax) of Ce6-AuNP-Lf irradiated with 4 W/cm² intensity PTT laser for 240 minutes was 64.5±4.5° C., whereas the Tmax of Ce6-AuNP was 43.0±3.7° C., and AuNP, which is vulnerable to aggregation, showed poor heat generation as in Ce6-AuNP.

That is, it could be confirmed that Ce6-AuNP-Lf showed the highest PTT efficiency despite bonding of AuNP with the Lf protein and Ce6 photosensitizer. Further, PEGylated Lf-conjugated AuNP (AuNP-Lf) also showed higher PTT efficiency, confirming that Lf protein conjugation did not affect the PTT efficiency of AuNP itself. That is, it could be confirmed that steric stabilization by PEGylation of AuNP has a significant effect on PTT efficiency.

1-3. Examination of Hydrophobicity of Nanoparticle Complex for Oral Administration

It was intended to examine the hydrophobicity of a simple photosensitizer (Ce6), the gold nanoparticles bonded to the photosensitizer (Ce6-AuNP) of Preparation Example 3, and the nanoparticle complex for oral administration (Ce6-AuNP-Lf) of Preparation Example 4.

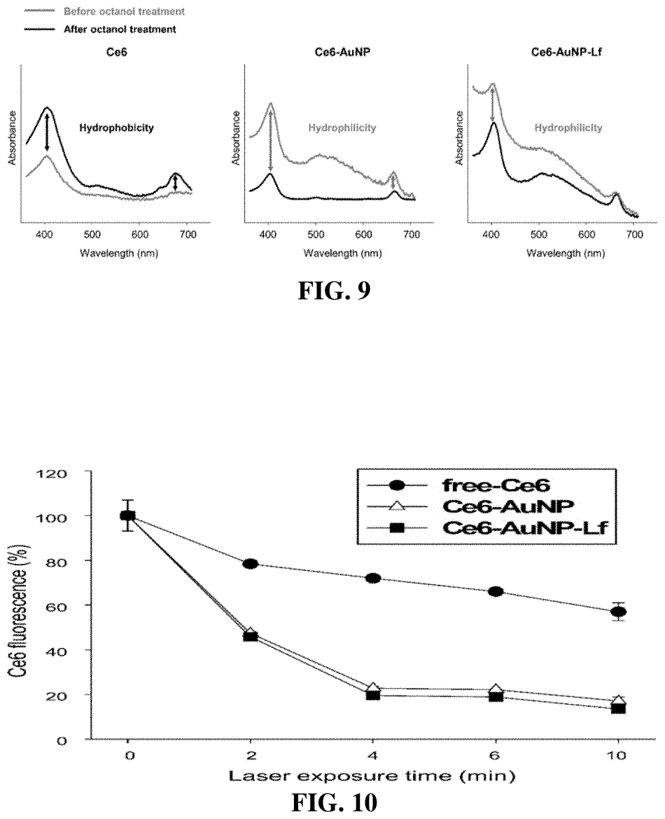

Specifically, absorbance at 400 nm and 671 nm was measured when Ce6, Ce6-AuNP, and Ce6-AuNP-Lf were dissolved in DW (600 μL) and octanol, which has strong hydrophobicity, was added to cause a phase transition, and the results are illustrated in FIG. 9.

FIG. 9 is a set of graphs showing the hydrophobic assay of a simple photosensitizer (Ce6), gold nanoparticles to which a photosensitizer is bonded (Ce6-AuNP) and a nanoparticle complex for oral administration (Ce6-AuNP-Lf).

Referring to FIG. 9, it could be confirmed that Ce6-AuNP and Ce6-AuNP-Lf exhibited hydrophilicity despite conjugation with hydrophobic Ce6. This could confirm that AuNP conjugation conferred hydrophilicity that could potentially facilitate mucus penetration of the mucus barrier. Conversely, since it could be confirmed that the mucin protein forms hydrophobic bonds with NPs during oral absorption, it could be confirmed that the surface hydrophobicity of NPs promotes immune recognition by macrophages, allowing the hydrophilic Ce6-AuNP-Lf to reduce undesirable immunostimulation.

Therefore, it could be confirmed that Ce6-AuNP-Lf is expected to be a promising Ce6 delivery carrier to enhance the accumulation of hydrophobic Ce6 in glioblastoma multiforme (GBM), which is a brain tumor tissue, through oral absorption.

### Example 2. Examination of Metal-Enhanced Reactive Oxygen Generation (MERos) of Nanoparticle Complex for Oral Administration

2-1. Examination of Metal-Enhanced Reactive Oxygen Generation (MERos) of Nanoparticle Complex for Oral Administration

Photobleaching was measured to examine the metal-enhanced reactive oxygen generation (MERos) of the nanoparticle complex for oral administration.

Specifically, the fluorescence decays of a simple photosensitizer (Ce6), the photosensitizer (Ce6-AuNP) of Preparation Example 3, and the nanoparticle complex for oral administration (Ce6-AuNP-Lf) of Preparation Example 4 according to laser irradiation were measured using fluorescence spectroscopy (Thermo Scientific™ VLBL00D0, USA).

In this case, each sample (Ce6 equivalent concentration of 2.5 μM) was dissolved in ethanol and irradiated with a PDT laser (MRL-III-671, 671±1 nm, China) for 10 minutes, and then the photobleaching of Ce6 fluorescence (λ excitation/λ emission=400 nm/671 nm) of each sample was measured according to laser irradiation time, and the results are illustrated in FIG. 10.

Specifically, FIG. 10 is a graph showing the Ce6 photo-bleaching rate of a simple photosensitizer (free-Ce6), gold nanoparticles to which a photosensitizer is bonded (Ce6-AuNP) and a nanoparticle complex for oral administration (Ce6-AuNP-Lf) by irradiation with PDT.

Referring to FIG. 10, since it could be confirmed that during 10 minutes of PDT laser irradiation, the Ce6 fluorescence of free-Ce6, Ce6-AuNP and Ce6-AuNP-Lf gradually decreased to 57.0±5.6%, 17.1±2.5% and 13±2.6%, respectively, it could be confirmed that due to photobleaching, fluorescence decreased with light irradiation by a 4-fold larger change in Ce6-AuNP and Ce6-AuNP-Lf compared to free-Ce6.

It could be confirmed that this is due to metal enhanced fluorescence (MEF) by plasmon coupling between AuNP and Ce6, and it could be confirmed that this improves fluorescence intensity but induces photobleaching.

Through this process, since it could be confirmed that this phenomenon is caused by MEF rather than FRET of the distance-dependent energy transfer process between two fluorophores, and AuNPs do not act as fluorophores, unlike Ce6, it could be confirmed that metal nanoparticles such as Au, Ag, Cu, and Pt are more suitable as MEFs that increase the fluorescence intensity of fluorophores.

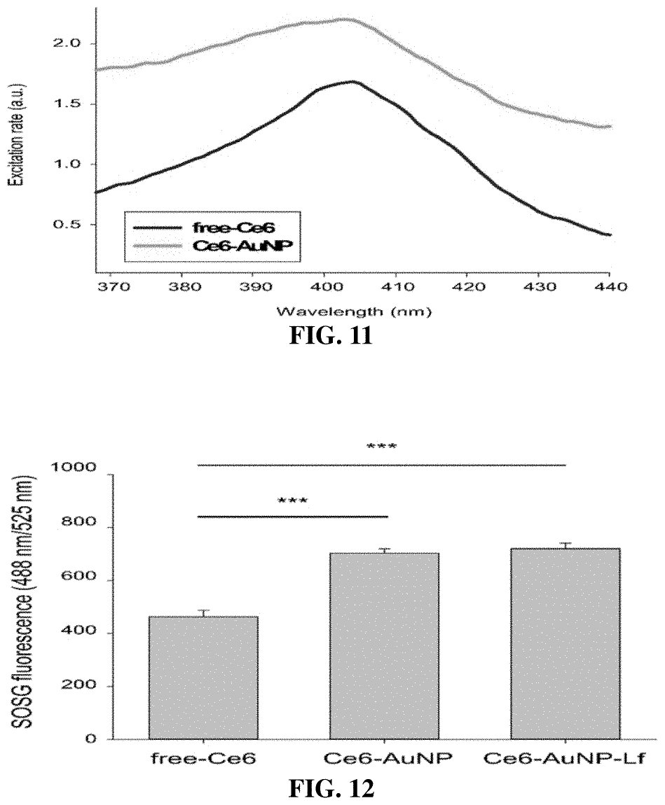

FIG. 11 illustrates the fluorescence excitation spectra of a simple photosensitizer (free-Ce6) and gold nanoparticles to which a photosensitizer is bonded (Ce6-AuNP).

Referring to FIG. 11, since it could be confirmed that Ce6-AuNP showed 1.3-fold improved excitation compared to free-Ce6 in the fluorescence excitation spectrum, it could be confirmed that AuNP may be used as a nanoantenna that increased the excitation energy of Ce6, which can either induce fluorescence or generate ROS.

In addition, in order to examine the relationship between the generated ROS and the decrease of Ce6 fluorescence, mixtures of the SOSG reagent (0.5 μM) and Ce6, Ce6-AuNP, and Ce6-AuNP-Lf (Ce6 equivalent concentration of 2.5 μM) were applied, and then the mixtures were irradiated with a PDT laser for 5 minutes, SOSG assay for detecting the generation of ROS was performed by measuring the production of singlet oxygen (¹O₂) at the fluorescence intensity (λ excitation/λ emission=488 nm/525 nm), and then the results are illustrated in FIG. 12.

FIG. 12 is a graph showing the SOSG analysis results of a simple photosensitizer (free-Ce6), gold nanoparticles to which a photosensitizer is bonded (Ce6-AuNP) and a nanoparticle complex for oral administration (Ce6-AuNP-Lf).

Referring to FIG. 12, since it could be confirmed that PDT irradiation generated 1.6-fold more ROS in the AuNP-conjugated Ce6 (Ce6-AuNP and Ce6-AuNP-Lf) than with free-Ce6, it was confirmed that ROS increased and Ce6 fluorescence decreased, and it could be confirmed that in AuNP-conjugated Ce6 such as Ce6-AuNP and Ce6-AuNP-Lf, MEF and ROS generation are characterized by simultaneous increases.

That is, it seems that the proximity of Ce6 to the gold ions on the AuNP surfaces enhanced spin-orbit coupling due to the external heavy atom effect, increasing triplet formation, so that the mechanism of the relationship between MEF and MERos could be expected to be due to inter-system crossover between AuNP and PS, which promotes the triplet state of the photosensitizer (PS).

2-2. Examination of Metal-Enhanced Reactive Oxygen Generation (MERos) of Nanoparticle Complex for Oral Administration According to PDT and PTT Application Order

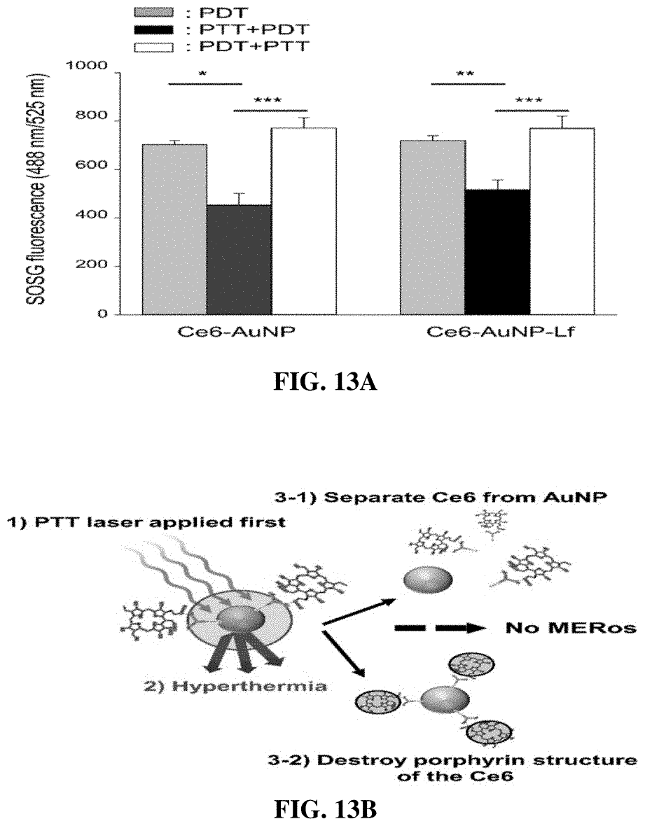

To examine the metal-enhanced reactive oxygen generation (MERos) of the nanoparticle complex for oral administration according to the PDT and PTT application order, PDT and PTT to mixtures of SOSG reagent (0.5 μM) along with Ce6, Ce6-AuNP, and Ce6-AuNP-Lf (Ce6 equivalent concentration of 2.5 μM) were applied, and then single laser groups (PTT or PDT) were irradiated for 5 minutes, and groups of a combination of lasers (PTT+PDT or PDT+PTT) were irradiated for 2.5 minutes for each laser to determine the generation of singlet oxygen (¹O₂) at the fluorescence intensity (λ excitation/λ emission=488 nm/525 nm), thereby performing SOSG assay for detecting the generation of ROS, and then the results are illustrated in FIG. 13.

FIG. 13A is a graph showing the SOSG analysis results of gold nanoparticles to which a photosensitizer is bonded (Ce6-AuNP) and a nanoparticle complex for oral administration (Ce6-AuNP-Lf) according to the application order of PDT and PTT.

Referring to 13A, it could be confirmed that Ce6-AuNP and Ce6-AuNP-Lf showed a significant difference in ROS generation ability according to the PDT and PTT application order, and as a result, it could be confirmed that PTT+PDT (application of PTT after application of PDT) generated the lowest ROS, whereas single PDT and PDT+PTT (application of PTT after application of PDT) were rather effective.

FIG. 13B is a schematic view illustrating a mechanism that may occur when PTT+PDT (application of PDT after application of PTT) is applied to the nanoparticle complex for oral administration.

Referring to FIG. 13B, it can be inferred that in the case of PTT+PDT (application of PDT after application of PTT), ROS generation is low due to the following mechanism. That is, AuNPs generate high heat in response to the first applied PTT laser, and accordingly, Ce6 may separate from the AuNP surface or destroy the porphyrin structure of Ce6, and as a result, since MERos is no longer present due to loss of plasmon coupling between Ce6 and AuNP, it could be predicted that ROS generation of the PTT+PDT laser group may be relatively lower than that of single PDT or the PDT+PTT laser group.

### Example 3. Examination of Stability of Nanoparticle Complex for Oral Administration in Oral Absorption Environment

It was confirmed whether the nanoparticle complex for oral administration is stable even in the oral absorption environment.

Specifically, in order to evaluate the colloidal stability of the nanoparticle complex for oral administration (Ce6-AuNP-Lf) in the oral absorption process, the stability of the nanoparticle complex for oral administration was evaluated after the pH environments of the stomach and intestinal system were mimicked.

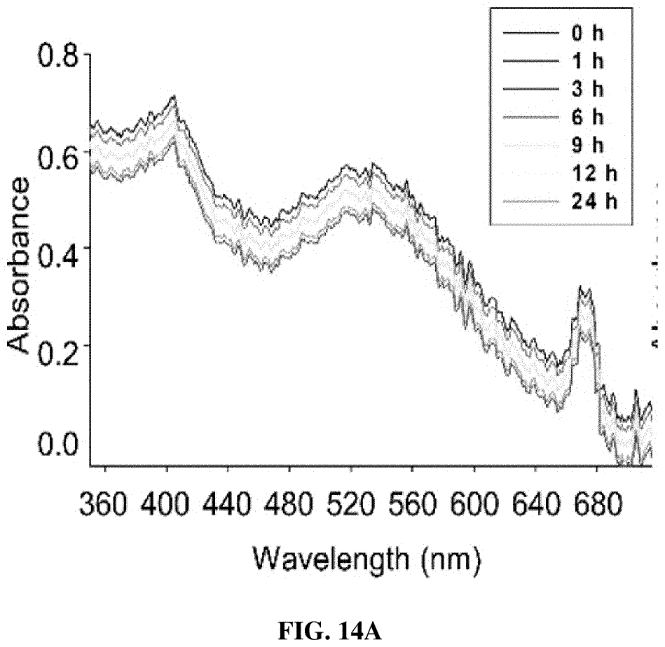

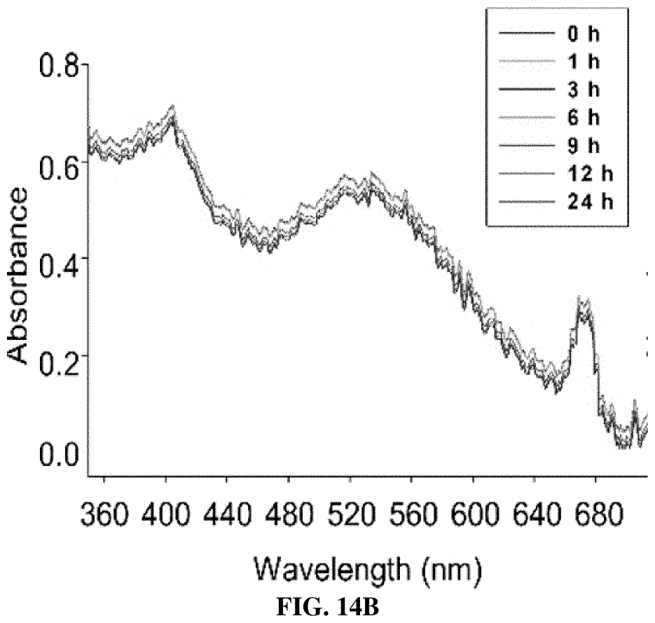

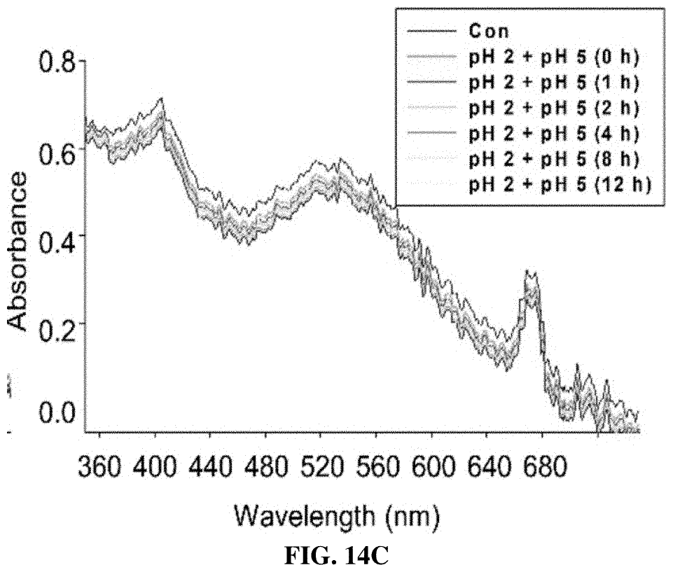

FIGS. 14A to 14C are UV-vis absorbance graphs of Ce6-AuNP-Lf under conditions mimicking the gastrointestinal microenvironment to which a material is exposed when orally administered (FIG. 14A, pH 2 for 24 hours; FIG. 14B, pH 5 for 24 hours; and FIG. 14C, pH 2 for 3 hours and changed to pH 5 for an additional 12 hours).

Referring to FIG. 14A, it could be confirmed that at pH 2, the absorbance of Ce6-AuNP-Lf was slightly decreased at 532 and 671 nm, but there was no peak shift.

Referring to FIG. 14B, it could be confirmed that at pH 5, no peak shift or decrease in absorbance was observed until 24 hours

Further, referring to FIG. 14C, it could be confirmed that to more specifically simulate the oral absorption procedure of Ce6-AuNP-Lf, when it was serially exposed to pH 2 for 3 hours and then pH 5 for an additional 12 hours, Ce6-AuNP-Lf showed stability without a peak shift and decreased absorbance at both the 532 and 671 nm wavelength bands.

Through this, it could be confirmed that Ce6-AuNP-Lf maintained the colloidal stability of AuNP and undesired release of Ce6 was not detected at the pH conditions of the GI tract.

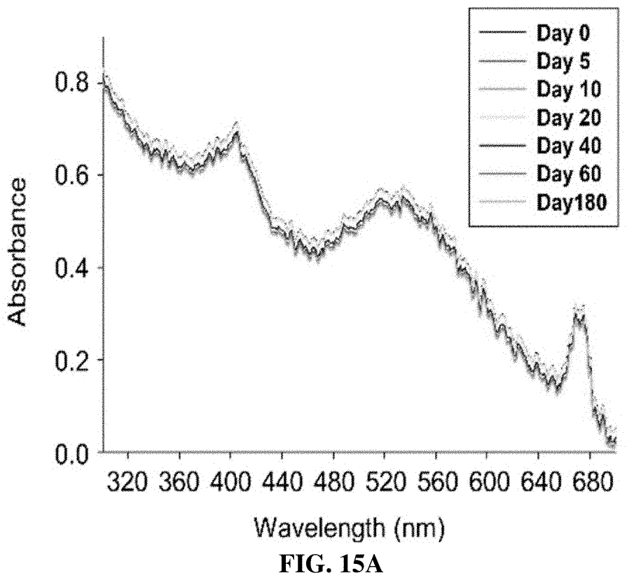

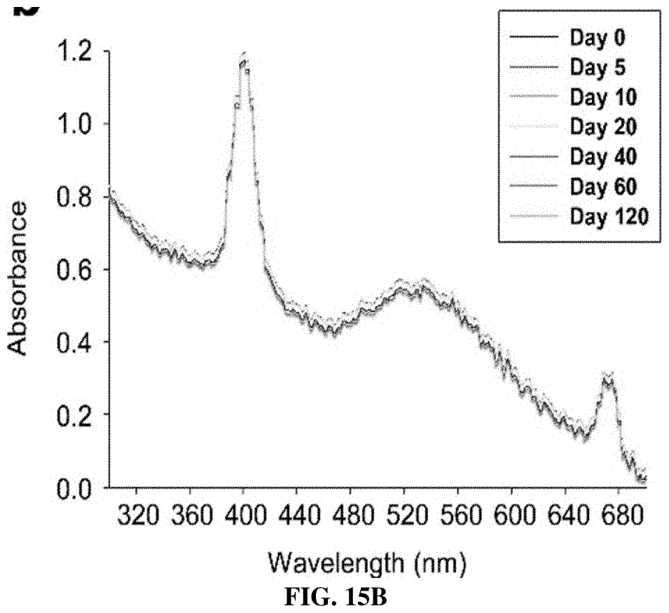

FIGS. 15A and 15B are UV-vis absorbance graphs of Ce6-AuNP-Lf in PBS (FIG. 15A) and 10% FBS (FIG. 15B) for 180 days.

Referring to FIGS. 15A and 15B, it could be confirmed that Ce6-AuNP-Lf was stable in physiological solutions of PBS and of 10% FBS for 180 days.

Based on these findings, it could be confirmed that Ce6-AuNP-Lf is suitable as an oral formulation.

### Example 4. Examination of Permeability of Nanoparticle Complex for Oral Administration in Small Intestinal Epithelial Barrier

4-1. Evaluation of Cytotoxicity of Nanoparticle Complex for Oral Administration

The nanoparticle complex for oral administration (Ce6-AuNP-Lf) targets a lactoferrin receptor present in the small intestine and enters the blood through the small intestine endothelial cells, and then, Ce6-AuNP-Lf present in the blood is targeted to brain tumor tissue and accumulated in brain tissue or blood vessels. Accordingly, in the present example, cytotoxicity experiments were performed on small intestine endothelial cells and vascular endothelial cells

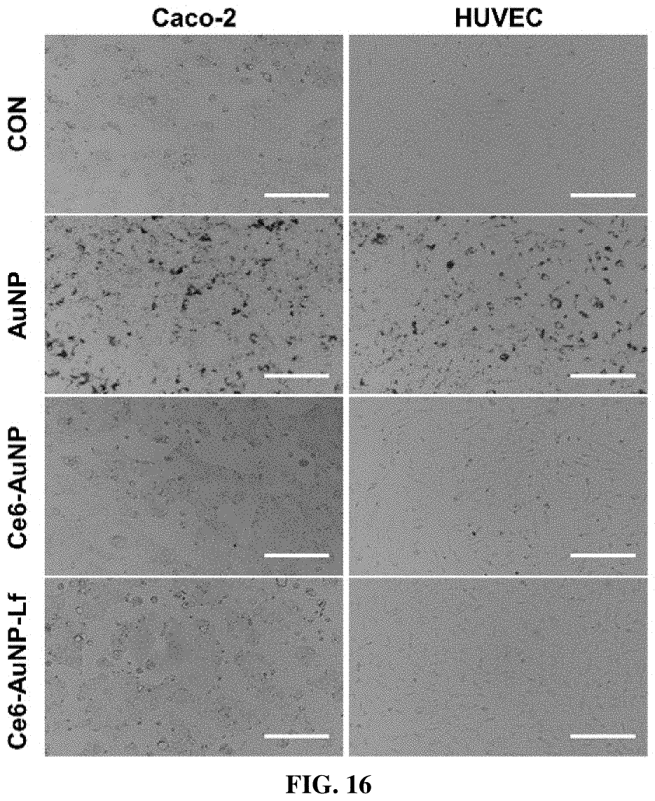

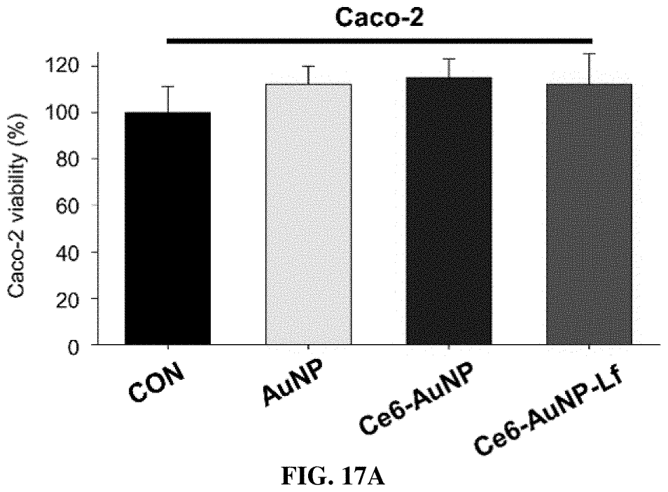

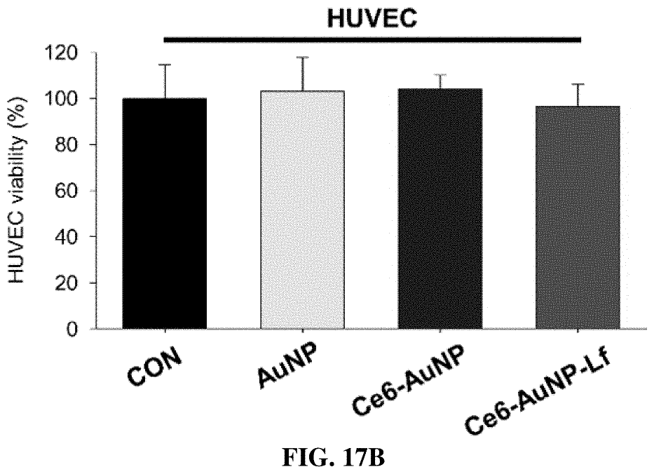

Specifically, human umbilical vein endothelial cells (HUVECs) and small intestine endothelial cells (Caco-2) were seeded in 96-well plates at a seeding density of 5×10³ cells for each well and incubated for 24 hours in a CO₂ incubator. Next, AuNP, Ce6-AuNP and Lf-PEG-AuNP (gold equivalent concentration of 10 μM) were applied for 24 hours. Next, after washing with PBS buffer, wells were treated with a culture medium containing EZ-Cytox at 37° C. and 5% CO₂ for 4 hours. In this case, the absorbance of the medium was measured with a micro-well plate reader at a wavelength of 450 nm, and the results are illustrated in FIGS. 16, 17A and 17B.

FIG. 16 illustrates TEM images showing the morphology of human vascular endothelial cells (HUVECs) and small intestine endothelial cells (Caco-2) treated with AuNP, Ce6-AuNP, and Lf-PEG-AuNP.

FIGS. 17A and 17B are graphs showing the cell viability of small intestine endothelial cells (Caco-2) (FIG. 17A) and human vascular endothelial cells (HUVECs) (FIG. 17B) treated with AuNP, Ce6-AuNP, and Lf-PEG-AuNP for 24 hours.

Referring to FIGS. 16, 17A, and 17B, since cytotoxicity and morphological changes were not detected in Caco-2 cells and HUVECs even after treatment with AuNP, Ce6-AuNP, and Lf-PEG-AuNP, it could be confirmed that the cytotoxicity was not exhibited.

4-2. Measurement of Small Intestine Endothelial Cell Permeability of Nanoparticle Complex for Oral Administration

In the present example, Caco-2 cell permeability assay was performed to confirm the oral absorption rate of the nanoparticle complex for oral administration in small intestine endothelial cells in vitro.

Specifically, small intestine endothelial cells (Caco-2) were inoculated onto a Transwell insert with a diameter of 6.5 mm and a pore size of 0.4 m (seeding density: 2×10⁴ cells/insert; Corning, Inc., Corning, N.Y., USA).

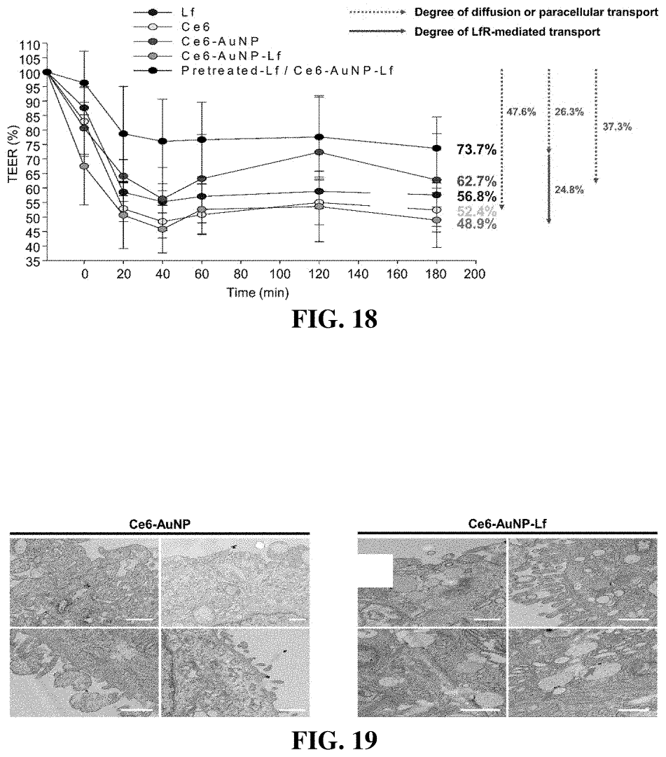

After the Caco-2 were incubated for approximately 2 to 3 weeks in a CO₂ incubator, trans epithelial electrical resistance (TEER) was measured using a voltmeter (EVOM²; World Precision Instruments, Sarasota, Fla., USA) to confirm tight junctions. In this case, the TEER value >3,700 Ω·cm² was used for the assay. Caco-2 cells were treated with Ce6, Ce6-AuNP, and Ce6-AuNP-Lf (gold equivalent concentration of 5 μM and Ce6 equivalent concentration of 2.5 μM). For competitive binding, free-Lf (5 μM) was pretreated for 2 hours, followed by Ce6-AuNP-Lf (gold equivalent concentration of 5 μM). During the treatment, TEER values were measured at each designated time, and the results are illustrated in FIG. 18.

FIG. 18 is a graph showing TEER values obtained through a cell monolayer after treating small intestine endothelial cells (Caco-2) with free-Lf, Ce6, Ce6-AuNP, Ce6-AuNP-Lf, and pretreated-Lf/Ce6-AuNP-Lf for 24 hours.

Referring to FIG. 18, it could be seen that the TEER values of the Ce6, Ce6-AuNP and Ce6-AuNP-Lf groups decreased to 52.4%, 62.7% and 48.9%, respectively, and decreased according to the drug permeability in the Caco-2 cell monolayer

Through this, transmittance of hydrophobic Ce6 (about 47.6%) may be interpreted as the result of intercellular diffusion into the cellular plasma membrane. Meanwhile, it could be confirmed that hydrophilic Ce6-AuNP and Ce6-AuNP-Lf were also transported across the barrier in amounts of 37.3% and 51.1%, respectively. Through this, it could be confirmed that Ce6-AuNP and Ce6-AuNP-Lf were able to cross the Caco-2 cell barrier through the paracellular pathway in the tight junctions.

FIG. 19 illustrates monolayer TEM images of small intestine endothelial cells (Caco-2) treated with Ce6-AuNP and Ce6-AuNP-Lf.

Referring to FIG. 19, since it could be confirmed that intracellular entrapment of Ce6-AuNP-Lf in vesicles was observed, it could be confirmed that lactoferrin receptor-mediated transcytosis (LfR-mediated transcytosis) occurred in Caco-2 cells, and referring to FIG. 18, it could be confirmed that the enhanced permeability of Ce6-AuNP-Lf (about 13% permeability) compared to Ce6-AuNP was due to the LfR-mediated transcytosis.

That is, since intestinal epithelial cells including Caco-2 cells express lactoferrin receptors (LfRs) on their membranes, it could be confirmed that the permeability of Ce6-AuNP-Lf in the gastrointestinal tract was increased by the lactoferrin receptors (LfRs).

4-3. Examination of Passive Transport of Nanoparticle Complex for Oral Administration to Small Intestine Endothelial Cells

Pretreated-Lf/Ce6-AuNP-Lf was used to examine passive transport excluding lactoferrin receptor (LfR)-mediated transport.

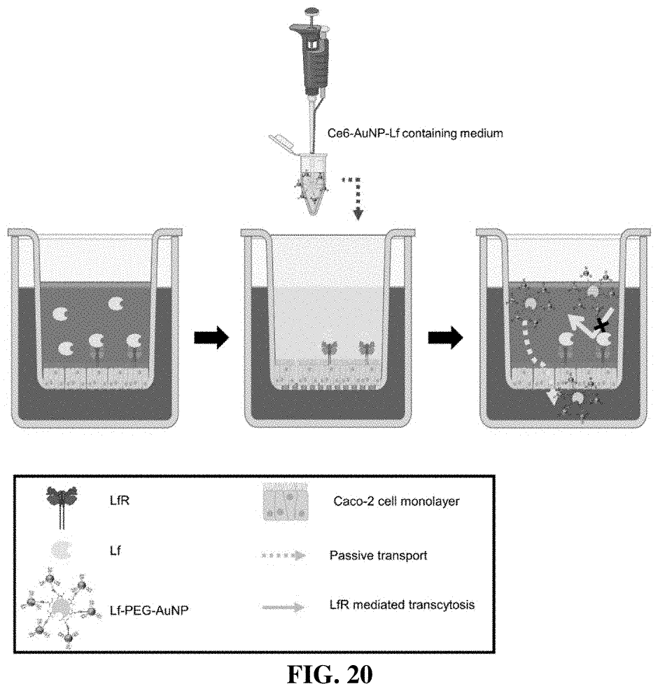

FIG. 20 is a schematic view of a pretreated-Lf/Ce6-AuNP-Lf group for confirming only passive transport of Ce6-AuNP-Lf in a Caco-2 cell monolayer.

Referring to this, Lf pretreatment (pretreated-Lf) was performed to saturate LfR expressed in the Caco-2 cell monolayer, and two hours after Lf pretreatment (pretreated-Lf), the medium was exchanged with a Ce6-AuNP-Lf-containing medium. Thereafter, when the results are examined, since it can be confirmed that Ce6-AuNP-Lf could not be subjected to transcytosis through LfR due to pretreated-Lf, it could be confirmed that that Ce6-AuNP-Lf penetrated the Caco-2 cell monolayer only through passive transport between tight junctions, and it could be confirmed even through FIG. 19 that the difference in transmittance between the pretreated-Lf/Ce6-AuNP-Lf treatment group and the Ce6-AuNP-Lf treatment group was 24.8%. That is, it could be confirmed that 24.8% of Ce6-AuNP-Lf is transported through LfR-mediated transcytosis and the remaining 26.3% is passively transported through an LfR-independent pathway, as with Ce6-AuNP.

### Example 5. Examination of Permeability and Photothermal Therapeutic Effect of Nanoparticle Complex for Oral Administration in Blood-Brain Barrier

5-1. Measurement of Blood-Brain Barrier Permeability of Nanoparticle Complex for Oral Administration

In the present example, a human BBB Transwell model was used to confirm the oral absorption rate of the nanoparticle complex for oral administration in vitro at the blood-brain barrier (BBB).

Specifically, human BMVECs were generated from human iPSCs as previously described by modification of oxygen conditions to mimic the hypoxic microenvironment of the developing brain.

A human iPSC line IMR90-4 (WiCell Research Institute) was maintained according to the WiCell Feeder Independent Pluripotent Stem Cell Protocol provided by the WiCell Research Institute.

IMR90-4 iPSCs were singularized using Accutase™ and seeded on a 6-well plate coated with Corning Matrigel® at a density of 1.7×10⁴ cells per well in the presence of Y27632 (10 M, Tocris Bioscience).

Next, cells were cultured with TeSR™-E8™ (STEMCELL Technology) for 3 days until the cell density reached 3×10⁵ cells per well (DO-D3).

To initiate differentiation into endothelial cells and neural progenitor cells, IMR90-4 iPSCs were switched from TeSR™-E8™ to unconditioned media (UM). In this case, the UM consisted of DMEM/F12 (78.5 mL), Knockout™ serum replacement (20 mL), non-essential amino acids (1 mL, 100×), GlutaMAX™ supplement (0.5 ml), and β-mercaptoethanol (182 μL) supplemented with CHIR-99021 (6 μM).

On the next day, the cell culture media were switched to UM supplemented with 1×B-27 supplement without CHIR-99021 and were changed daily for 5 days (D4-D9).

For the next 2 days (D9-D11), the endothelial cells were selectively expanded by changing to endothelial cell media (EC).

The endothelial cell media were human endothelial SFM supplemented with 20 ng/mL of basic fibroblast growth factor, 1×B-27 supplement, and retinoic acid (RA, 10 μM).

On day 11, cells were harvested from the 6-well plates using Accutase™ and seeded on a 0.4 m pore-sized 24-well Transwell insert chamber coated with collagen IV (400 μg/mL) and fibronectin (100 μg/mL) at a density of 3.3×10⁴ cells per insert.

Then, to recapitulate the phenotypic features of BBB, BMVECs inoculated onto the insert chamber were cultured with a mixture of human primary astrocytes (ScienCell) and pericytes (ScienCell) in the basal chamber.

On day 12, media were switched to EC without bFGF and RA and changed daily to maintain the BBB culture.

From D6-D12, cells were cultured in a flushed hypoxic incubator (EppendorfGalaxy® 48R) with a 5% O₂-5% CO₂—N₂ balance and transferred to a regular CO₂ incubator.

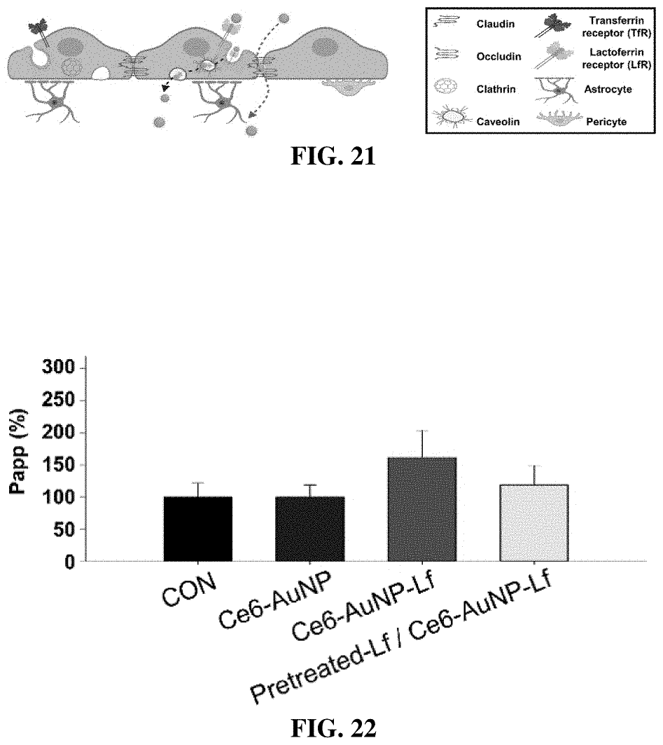

FIG. 21 is a schematic view of a human BBB Transwell model.

Referring to the drawing, it could be confirmed that the BBB Transwell model was prepared by culturing human brain microvascular endothelial cells on the apical side of the insert connected with primary human pericytes and astrocytes.

The BBB Transwell model was prepared and a TEER value, which is an indicator of development of tight junctions, was measured to determine the blocking function of BBB.

Specifically, as a result of measuring the impedance values for the BBB using a TEER measurement machine (EVOM², World Precision Instruments), the BBB endothelium showed a physiological level of the TEER value (average 4079 Ω·cm²), confirming that the BBB model can provide very limited paracellular transport.

The amount of AuNPs that crossed the BBB was measured using the prepared human BBB Transwell model.

Specifically, the human BBB model prepared above with TEER values >3,700 Ω·cm² was used for the assay, and FITC-Dextran 3 kDa was used to monitor the barrier integrity of the BBB during the assay.

The media were changed to human endothelial SFM media at 2 hours before the assay, and Ce6-AuNP and Ce6-AuNP-Lf (gold equivalent concentration of 5 μM) were applied to the apical side of an in vitro BBB system in the absence of FITC-Dextran 3 kDa (250 μg/mL).

The Transwell plates were incubated at 37° C. with stirring, and samples (200 μL) from the basal chamber were collected every 30 minutes for 2 hours while adding the same volume of human endothelial SFM media to the basal chamber.

The quantification of nanoparticles in the basal chamber was performed using an inductively coupled plasma mass spectrometer (ICP-MS, iCAP RQ; Thermo Fisher Scientific, USA) and the fluorescent intensities of the samples at excitation wavelength of 495 nm and emission wavelength of 519 nm were measured. The apparent permeability (Papp) was analyzed using a micro-plate reader (Thermo Scientific™ VLBL00D0, USA).

In this case, the Papp of NP and FITC-Dextran was calculated as follows.

$${P}_{app} = \frac{{V}_{b} \times {C}_{t}}{{C}_{0} \times Δ t \times A} \qquad [ Equation 1 ]$$

*Vb is the volume of basolateral chamber, Cₜ is the change in concentration, Δt is change in time at steady state, A is the growth area (0.33 cm² in the 24-well Transwell), and C₀ is the initial concentration in the apical chamber.

Further, to determine the endocytosis mechanism of Ce6-AuNP-Lf, the change in the Papp of Ce6-AuNP-Lf was determined in the presence of chlorpromazine hydrochloride, which is a clathrin-mediated endocytosis blocker.

After pre-treatment with chlorpromazine hydrochloride (50 μM) for 2 hours, Ce6-AuNP-Lf (5 μM) was added to the apical chamber of the in vitro BBB system in the presence of an inhibitor.

In addition, to identify the receptor specificity of Ce6-AuNP-Lf, the in vitro BBB system was pre-treated with Lf (5 μM) for 2 hours.

After pre-treatment with Lf for 2 hours, Ce6-AuNP-Lf (5 μM) was added to the apical chamber of the in vitro BBB system in the presence of pre-treated Lf.

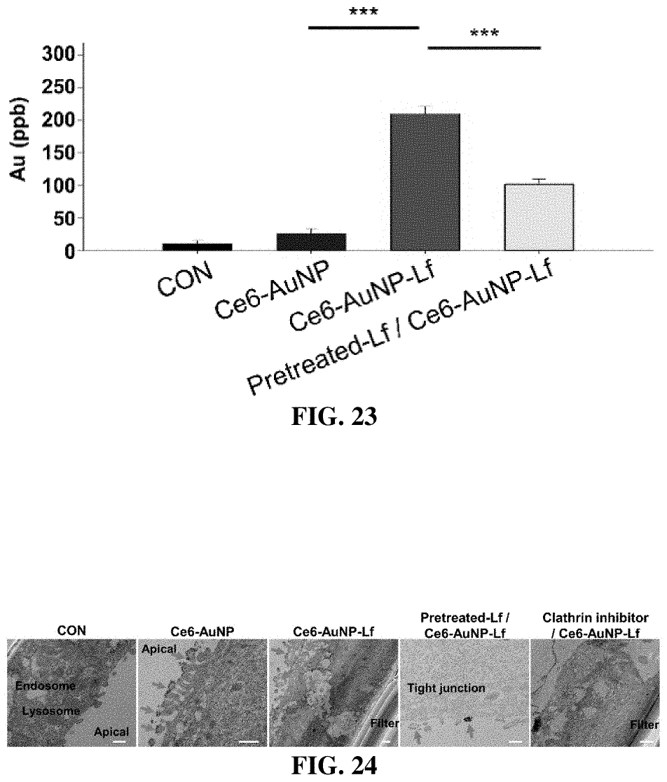

FIG. 22 is a graph showing an apparent permeability (Papp) of a control (Con), Ce6-AuNP, Ce6-AuNP-Lf, and pretreated-Lf/Ce6-AuNP-Lf using FITC-dextran.

FIG. 23 is a graph showing the absolute amount of gold nanoparticles permeating the BBB model of a control (Con), Ce6-AuNP, Ce6-AuNP-Lf, and pretreated-Lf/Ce6-AuNP-Lf measured by ICP-MS.

Referring to FIG. 22, the apparent permeability (Papp) of FITC-Dextran (3 kDa) could be measured to evaluate the integrity of tight junctions.

Furthermore, referring to FIG. 23, the concentration of Ce6-AuNP-Lf in the basal chamber of Transwell was 8.13-fold higher than the concentration of Ce6-AuNP, confirming that functionalization using Lf significantly enhanced BBB penetration efficacy without disrupting the barrier function. Further, pretreatment of Lf for the BBB model decreased BBB penetration of Ce6-AuNP-Lf by 52%, confirming that BBB penetration of Ce6-AuNP-Lf is dependent on the interaction between Lf and LfR.

FIG. 24 illustrates TEM images of the BBB model treated with a control (Con), Ce6-AuNP, Ce6-AuNP-Lf, pretreated-Lf/Ce6-AuNP-Lf, and Clathrin inhibitor/Ce6-AuNP-Lf.

Specifically, for bio-TEM images of the Transwell-cultured human BBB model, the samples were fixed after 2 hours of exposure according to the experimental conditions for each group (Ce6-AuNP, Ce6-AuNP-Lf, pretreated-Lf/Ce6-AuNP-Lf, and Clathrin inhibitor/Ce6-AuNP-Lf). In this case, reagent setup and the procedure followed the reported method. Next, a 4% PFA solution was added to the apical and basolateral chambers of the Transwell filters (0.5 and 1.5 mL, respectively) for 1 hour. Sorensen's phosphate buffer, which consists of solutions A and B (A is 0.2 M Na₂HPO₄→2H₂O, B is 0.2 M NaH₂PO₄→H₂O), was added for 10 minutes to rinse off the fixative. Next, 1% osmium tetroxide (OsO₄) was added to stain the cell monolayer for 1 hour. Next, Sorensen's phosphate buffer was used to wash the remaining OsO₄ for 10 minutes. Dehydration of cell monolayers on both sides of the filters was performed with different concentrations of ethanol as follows: 30% for 10 minutes, 50% for 10 minutes, 70% for 10 minutes, 90% for 10 minutes, and finally 100% for 20 minutes three times. The method of forming an epoxy resin block with a Low Viscosity Embedded Media Spurr's Kit method was applied to the entire Transwell containing the fixed cell monolayer. The epoxy resin block was cut to 4 mm, and the surrounding plastic and resin were removed by additional cutting to reveal the cubic alignment including plastic, epoxy resin, filter membrane, epoxy resin and the like from left to right. The specimens were cut into 80 nm-thick sections using a microtome and the obtained sections were air-dried for at least 1 hour. Copper grids were mounted in 2% uranyl acetate for 20 minutes, briefly washed with DW and mounted in lead citrate (0.4%) for staining for 10 minutes. Next, the section placed on the grid was observed using an 80-kV transmission electron microscope.

Referring to FIG. 24, it could be confirmed that Ce6-AuNP-Lf was internalized by brain endothelial cells packed by vesicles implying the LfR-mediated endocytosis of Ce6-AuNP-Lf. Further, it could be confirmed that Ce6-AuNP-Lf is not internalized by brain endothelial cells in the presence of Lf. Meanwhile, as a result of examination in the presence of the inhibitor of clathrin-mediated endocytosis to investigate the endocytic mechanism, it could be confirmed that Ce6-AuNP-Lf was still detected inside the vesicles despite the blocking of clathrin-mediated endocytosis, confirming that Ce6-AuNP-Lf can be internalized by cells through clathrin-independent endocytosis.

5-2. Examination of Brain Tumor Targeting Enhancement Effect of Nanoparticle Complex for Oral Administration

In the present example, the absorption rate of the nanoparticle complex for oral administration to brain tumor cells (U87MG) was confirmed in vitro.

Specifically, brain tumor (U87MG) cells (seeding density: 5×10⁴ cells/well) in a 4 well Nunc™ Lab-Tek™ II Chamber Slide™ System (Thermo Scientific™ 154526PK, USA) were treated with Ce6, Ce6-AuNP, or Ce6-AuNP-Lf (Ce6 equivalent concentration of 2.5 μM) for 18 hours, and then after washing three times with PBS, the cells were fixed with 4% PFA for 15 minutes. Next, DAPI mounting medium (Vector Laboratories, Inc., Burlingame, Calif., USA) was used and the intracellular Ce6 fluorescence was observed under a confocal microscope (TCS SP5, Leica, Germany).

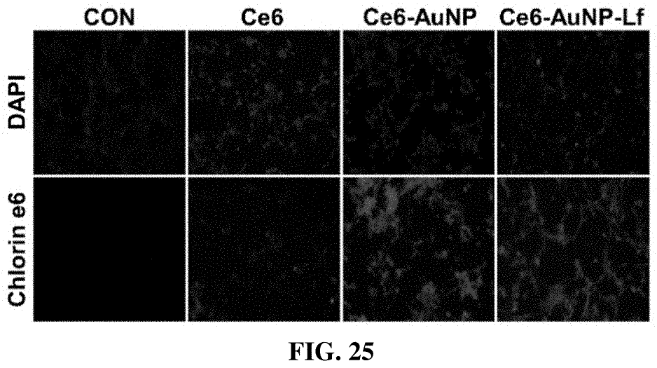

FIG. 25 illustrates confocal images after treating brain tumor cells (U87MG) with Ce6, Ce6-AuNP, and Ce6-AuNP-Lf, respectively.

Referring to the drawing, it could be confirmed that the intracellular fluorescence was significantly increased in the Ce6-AuNP and Ce6-AuNP-Lf groups compared to that of free-Ce6.

Meanwhile, flow cytometry (FACS Calibur™; BD Biosciences, Franklin Lakes, N.J.) was used to quantify the intracellular Ce6 fluorescence. Next, brain tumor (U87MG) cells (80% confluency in 100 π culture dish) were treated with Ce6, Ce6-AuNP, or Ce6-AuNP-Lf (Ce6 equivalent concentration of 2.5 μM) for 18 hours. After washing three times with PBS, the cells were detached with trypsin/EDTA, centrifuged for 3 minutes at 1000 rpm, and then analyzed with FACS.

As a result, it could be confirmed that the amounts of Ce6, Ce6-AuNP and Ce6-AuNP-Lf uptake into the cells were 43.6±2.2, 61.3±1.6 and 82.1±2.1, respectively.

That is, it could be confirmed that the cellular uptake of Ce6-AuNP-Lf was statistically increased compared to that of Ce6-AuNP, which lacks Lf as a targeting ligand, confirming that brain tumor cell (U87MG) uptake for targeted conjugates was enhanced compared to non-targeted conjugates.

5-3. Examination of Photothermal Therapeutic Effect of Nanoparticle Complex for Oral Administration

The photothermal therapeutic effect of the nanoparticle complex for oral administration was confirmed through cell cytotoxicity assay.

Specifically, brain tumor (U87MG) cells (seeding density: 5×10³ cells/well) were treated with Ce6, Ce6-AuNP, or Ce6-AuNP-Lf (5 μM of gold and Ce6 equivalent concentration of 2.5 μM) for 12 hours. Next, after washing three times with PBS, the cells were irradiated with a single laser (PTT or PDT) for 5 minutes or a combination of lasers (PTT+PDT or PDT+PTT) for 2.5 minutes each. Cell viability was measured with an EZ-Cytox kit.

Meanwhile, the intracellular ROS generation by laser irradiation was measured using the fluorescent probe DCFH-DA according to the manufacturer's instructions. Annexin V-DY-634/PI apoptosis was used to evaluate apoptosis. That is, brain tumor (U87MG) cells were suspended in 400 μL binding buffer, and then stained with Annexin V-DY-634 (5 μL) at 2 to 8° C. for 15 minutes. Next, PI (5 μL) was added and the cells were incubated for 5 min. In this case, brain tumor (U87MG) cells were analyzed using FACS.

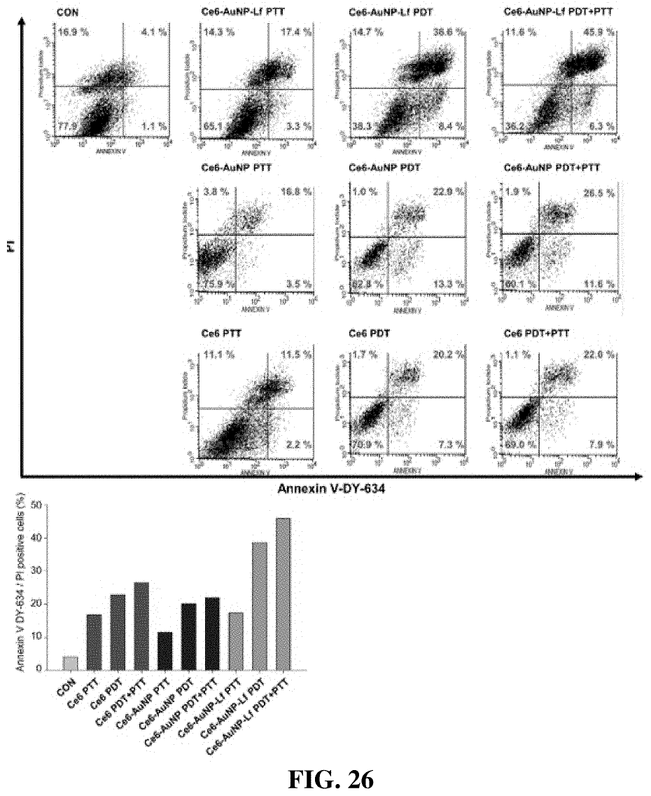

FIG. 26 is a set of graphs of Annexin V-DY-634/PI apoptosis staining (ab214484, UK) for detecting apoptosis generated by irradiating brain tumor (U87MG) cells with a laser.

Referring to the drawing, it could be confirmed that the apoptotic cell populations (Annexin-V+/PI+) of Ce6-AuNP-Lf, Ce6-AuNP and Ce6 groups to which PDT+PTT was applied were 45.9%, 26.5%, and 22.0% of the total, respectively, confirming that PDT+PTT (application of PTT after application of PDT) showed an excellent therapeutic effect compared to other treatment methods such as PTT+PDT (application of PDT after application of PTT) and single PDT or PTT.

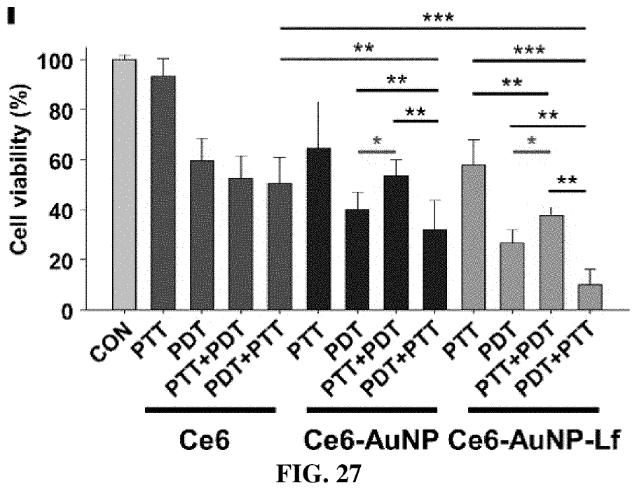

FIG. 27 is a graph showing cell viability after irradiating brain tumor (U87MG) cells with a laser.

Referring to FIG. 27, it could be confirmed that PTT+PDT (application of PDT after application of PTT) in the cells treated with Ce6-AuNP and Ce6-AuNP-Lf was less effective than single PDT.

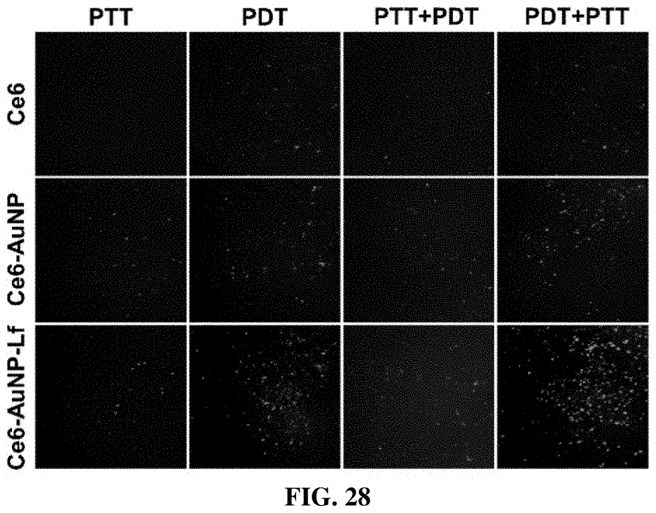

FIG. 28 is a set of images illustrating DCFH-DA fluorescence signals after irradiating brain tumor (U87MG) cells with a laser (excitation/emission at 485 nm/535 nm).

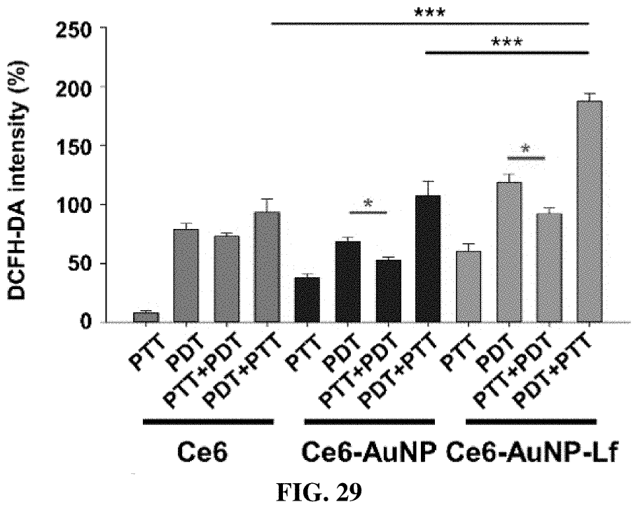

FIG. 29 is a graph showing the ROS production rate versus DCFH-DA fluorescence signals after irradiating brain tumor (U87MG) cells with a laser.

Referring to FIGS. 28 and 29, it could be confirmed that intracellular ROS fluorescence by the cells treated with Ce6-AuNP or Ce6-AuNP-Lf was lower in PTT+PDT (application of PDT after application of PTT) than in the single PDT but highest in PDT+PTT (application of PTT after application of PDT).

That is, it could be confirmed that when PTT was first applied, Ce6-AuNP and Ce6-AuNP-Lf, in which Ce6 is bonded to AuNP, lost the MERos effect, resulting in decreased ROS generation, confirming once again that the therapeutic effect of PTT+PDT (application of PDT after application of PTT) is lower than that of PDT+PTT (application of PTT after application of PDT) and PDT alone.

### Example 6. Examination of Absorption of Nanoparticle Complex for Oral Administration Using Animal Model

In the present example, it was intended to examine improved oral availability through an in vivo pharmacokinetics (PK) study.

Specifically, Balb/c mice were administered Ce6-AuNP-Lf at 30 mg/kg, 60 mg/kg, and 5 mg/kg via subcutaneous (SC), oral, and intravenous (IV) administration, respectively. Blood samples (500 μl) were collected by intra-cardiac puncture at each time point of 10 minutes, 20 minutes, 30 minutes, 60 minutes, 2 hours, 6 hours, and 12 hours after administration of Ce6-AuNP-Lf. In this case, the exact weights of blood samples were measured in a borosilicate glass tube.

Next, 70% nitric acid (800 μl) was added to each glass tube and samples were heated in a hot water bath at 60° C. for 3 hours. Next, HCl (37%) was added to each glass tube, and samples were heated under the same conditions. Next, the blood samples were transferred into 50 mL tubes and the pH was adjusted with 2% nitric acid and 0.5% HCl in DW. Next, the adjusted blood samples were filtered (0.22 μm pore-size) and analyzed by ICP-MS. The Au concentration was calculated and adjusted for sample weight. In this case, the standard curve of Au (0.0001 to 0.05 μg/mL) was linear, and the limit of detection was 0.0005 μg/mL. Background gold concentration, which is a measured pre-dose, was subtracted from measured values to derive a gold concentration attributed to the dose. In this case, the gold concentration in each sample was determined from the mean of six replicate measurements. Further, the bioavailability (BA) value was calculated from the following Equation 3.

$$BA ( F \% ) = \frac{{AUC}_{Oral or S . C}}{{AUC}_{IV}} \times \frac{{Dose}_{I . V}}{{Dose}_{Oral or S . C}} \qquad [ Equation 3 ]$$

* AUC_{oral or SC} is the area under curve in the case of oral or SC administration, respectively, AUC_{IV} is the area under curve in the case of IV administration, Dose_{oral or SC} is the dosage of drug injected orally or via SC administration, and Dose_{IV} is the dose of drug injected via IV administration.

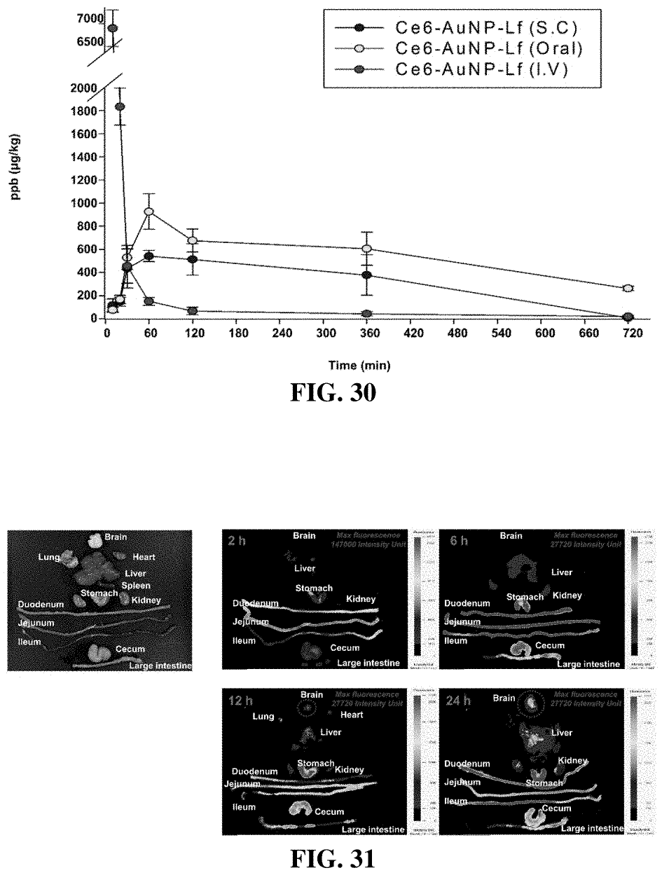

FIG. 30 is a graph showing the pharmacokinetic results of administering Ce6-AuNP-Lf to Balb/c mice at 30 mg/kg, 60 mg/kg and 5 mg/kg via subcutaneous (SC), oral, and intravenous (IV) administration, respectively.

TABLE 2

| | Dose | Tₘₐₓ | Cₘₐₓ | C₀ | V_{d} | T_{1/2} | AUC₀₋₇₂₀ | Fabs |
|---|---|---|---|---|---|---|---|---|
| Route | (mg/kg) | (min) | (μg/mL) | (μg/mL) | (mL/kg) | (min) | (μg min/kg) | (%) |
| Subcutaneous | 30 | 60 | 0.5 ± 0.1 | — | — | 395 ± 10 | 227,608 ± 3,254 | 8.6 ± 0.6 |
| (S.C.) | | | | | | | | |
| Oral | 60 | 60 | 0.9 ± 0.2 | — | — | 521 ± 20 | 385,450 ± 2,346 | 7.3 ± 1.2 |
| Intravenous | 5 | 0 | 62.5 | 62.5 | 80 | 5.6 ± 3.1 | 443,054 ± 4,410 | 100 |
| (I.V) | | | | | | | | |

* Periodic gold concentrations in blood are measured by ICP-MS after subcutaneous, oral and intravenous administration of Ce6-AuNP-Lf. Data is expressed as mean ± SEM (n = 3). AUC₀₋₇₂₀, area under the plasma concentration curve from 0 to 720 hours; Cₘₐₓ, maximum drug concentration in plasma; C₀, initial drug concentration; V_{d}, volume of distribution; F_{sbs}, absolute bioavailability; t_{1/2}, half-life; tₘₐₓ, time to Cₘₐₓ.

Referring to FIG. 30 and Table 2, it could be confirmed that the half-lives (T_{1/2}) of the oral administration group and the SC (subcutaneous) administration group were 521±20 minutes and 395±10 minutes, respectively. In addition, it could be confirmed that the residence time of Ce6-AuNP-Lf in blood was long, which was due to the short PEG (232 Da) coating on the surface of AuNP, and it could be confirmed that incorporation of PEG into the AuNPs enhanced the absorption of the nanoparticle complex into systemic circulation and increased its residence time in blood circulation. Furthermore, it could be confirmed that shorter PEG chains increased the absorption efficiency during oral administration compared to longer chains.

Further, SC injection is used as a sustained diffusible method among various drug injection routes, which is due to the large amount of capillaries in the fatty layer of subcutaneous tissue just beneath the skin. However, the mean residence time (MRT) of orally administered Ce6-AuNP-Lf was better than that of SC. In addition, it could be confirmed that the percentages of bioavailability (Fabs) were similar at 8.6±1.2% and 7.3±0.6% in the SC group and the oral group, respectively.

Furthermore, fluorescence signals were used to confirm whether orally administered Ce6-AuNP-Lf can be delivered to brain tumor tissue via systemic circulation.

Specifically, for the fluorescence tracer image, Balb/c mice were administered Ce6-AuNP-Lf at 60 mg/kg, and after 2, 6, 12, and 24 hours, the experimental mice were sacrificed, and the organs were extracted. In this case, the fluorescence signals of Ce6-AuNP-Lf in organs were imaged using an in vivo imaging system (FOBI, CELLGENTEK, Korea), and the exposure time was fixed to 550 seconds for analyzing fluorescence signals from tissues.

FIG. 31 illustrates a set of fluorescence tracer images of orally administered Ce6-AuNP-Lf.

In this case, intensity unit indicates the intensity/min/gain, and a red dashed line indicates the detected fluorescence signal in the brain.

Referring to FIG. 31, it could be confirmed that the fluorescence signal was maximum in the fluorescence tracer image of orally administered Ce6-AuNP-Lf at 2 hour with 147,000-intensity units (I.U.) in the jejunum, where LfR is overexpressed. Since the I.U. of the fluorescence decreased to 27,720 after 6 hours, it could be confirmed that the orally administered Ce6-AuNP-Lf was first delivered from the intestine to the liver before reaching systemic blood circulation, confirming that the fluorescence signal in the liver continued to increase up to 24 hours. Particularly, since it could be confirmed that the brain fluorescence signal was found after 12 hours and increased to about 13,000 I.U. at 24 hours after oral administration, it can be inferred that orally administered Ce6-AuNP-Lf is stably absorbed with high efficiency in the gastrointestinal tract. Next, through the previous example, since it could be confirmed that Ce6-AuNP-Lf can flow into the blood through the capillaries of the small intestine and overcome the BBB barrier to reach brain tissue, it could be confirmed that orally administered Ce6-AuNP-Lf has excellent pharmacokinetic properties and thus can be efficiently delivered to brain tumor tissue via systemic circulation.

### Example 7. Examination of Targeting Effect of Nanoparticle Complex for Oral Administration Using Animal Model

7-1. Examination of Brain Tumor Targeting Effect Using Brain Tumor Animal Model

In the present example, it was intended to confirm the brain tumor targeting effect of the nanoparticle complex for oral administration using a brain tumor animal model.

Specifically, PBS (containing 0.8×10⁶ C₆ glioma cells, 100 μL) was inoculated subcutaneously in the right flanks of athymic Balb/c nude mice. Next, five days after cell inoculation, the mice were randomly divided into 3 groups (n=4). A CON group received saline administration. Ce6-AuNP-Lf was administered via oral (gold equivalent concentration of 0.07 μM) and intravenous (IV) (gold equivalent concentration of 0.012 μM) methods, respectively.

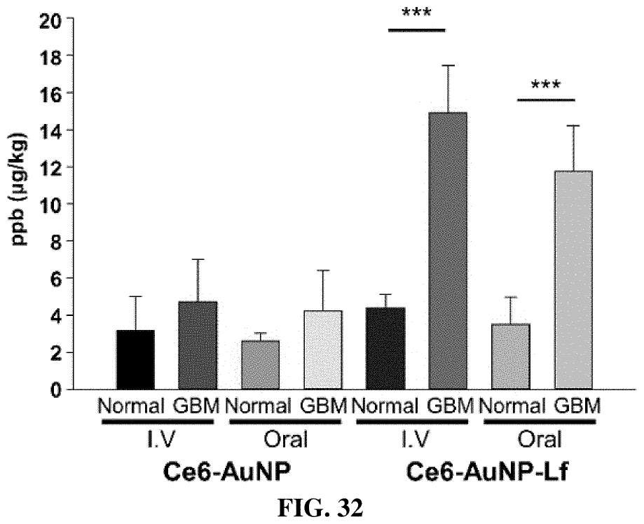

FIG. 32 is a graph showing AuNP accumulation rates in normal brain and GBM brain rat models 24 hours after intravenous (IV) and oral administration of Ce6-AuNP-Lf and Ce6-AuNP.

Referring to FIG. 32, it could be confirmed that IV administration of Ce6-AuNP-Lf showed an accumulation of 14.9±2.2 μg/kg and 4.3±0.6 μg/kg in GBM and normal brains, respectively, and during oral administration, Ce6-AuNP-Lf accumulated at 11.8±2.1 μg/kg in the GBM brain and at 3.5±1.3 μg/kg in the normal brain.

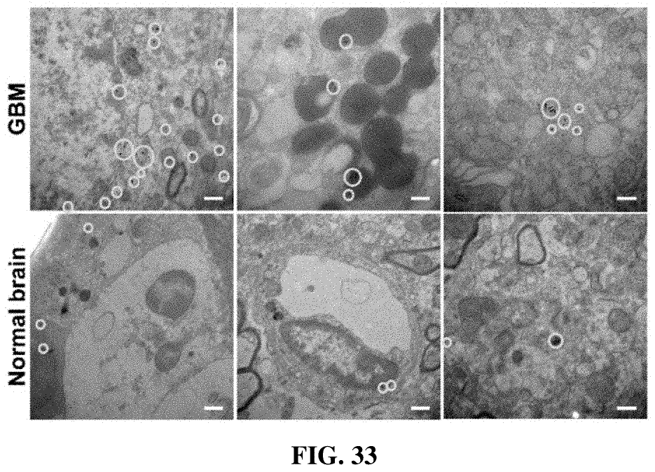

FIG. 33 is a set of bio-TEM images of a GBM brain and a normal brain after orally administering 60 mg/kg Ce6-AuNP-Lf.

Referring to FIG. 33, it could be confirmed that in the bio-TEM result of brain tissue collected 24 hours after oral administration, more Ce6-AuNP-Lf was found in the GBM compared to in normal brain regions.

7-2. In Vivo Experiment Using Subcutaneous GBM Xenograft Mouse Model

To additionally confirm the presence of GBM targeting and the non-specific drug distribution of Ce6-AuNP-Lf, in vivo experiments using a subcutaneous GBM xenograft mouse model were performed.

Specifically, PBS (containing 0.8×10⁶ C6 glioma cells, 100 μL) was inoculated subcutaneously in the right flanks of athymic Balb/c nude mice. Next, five days after cell inoculation, the mice were randomly divided into 3 groups (n=4). A CON group received saline administration. Ce6-AuNP-Lf was administered via oral (gold equivalent concentration of 0.07 μM) and intravenous (IV) (gold equivalent concentration of 0.012 μM) methods, respectively. Next, 24 hours after administration, the mice were irradiated sequentially with PDT and PTT lasers for 5 minutes. This treatment cycle was repeated three times and the survival rate, body weight, and tumor size were monitored until day 18. Tumor size was determined using calipers to measure the length a and width b of tumors and was calculated as 4/3π×α²×b² (a: smaller radius; b: larger radius). On day 18, the mice were sacrificed for further analysis.

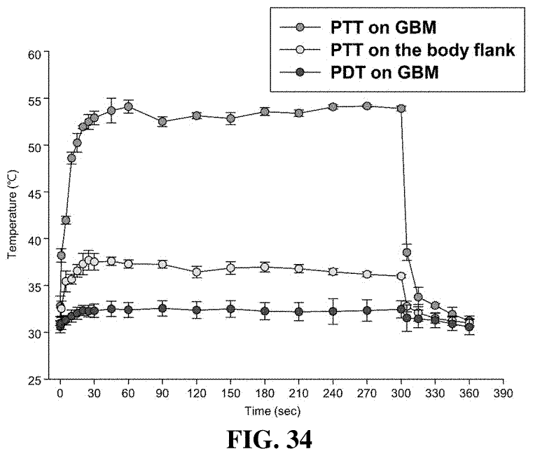

FIG. 34 is a graph confirming specific GBM targeting of Ce6-AuNP-Lf through thermal conversion characteristics under PTT laser after oral administration (60 mg/kg) for 24 hours.

Referring to FIG. 34, as a result of applying a PTT laser to the GBM site and the body flank after 24 hours of oral administration, an excellent targeting effect of the Ce6-AuNP-Lf with respect to GBM was identified through a temperature increase to 54.2±0.1° C., and simultaneously, the temperature rise in the body flank was 37.6±0.4° C. due to the reduction of the non-specific distribution by high targeting to GBM. Therefore, it could be confirmed that the GBM targeting of Ce6-AuNP-Lf is suitable for GBM therapy.

In order to clearly evaluate whether the transmitted PDT laser can have an energy level capable of generating ROS from AuNPs and Ce6-AuNP-Lf, the outside of mouse skin tissue (1 cm thickness) was irradiated with a PDT laser (671 nm).

Specifically, after 1 cm-thick biological tissue was extracted from mouse skin, 10 μL of a DPBF solution (10 mg/mL in DMSO) was added to Ce6, Ce6-AuNP, and Ce6-AuNP-Lf (Ce6 equivalent concentration of 10 μM) diluted with 1.5 mL of water. Next, the mouse skin tissue was irradiated with an NIR laser (671 nm) for 1, 3, and 5 minutes with or without the insertion of the biological tissue.

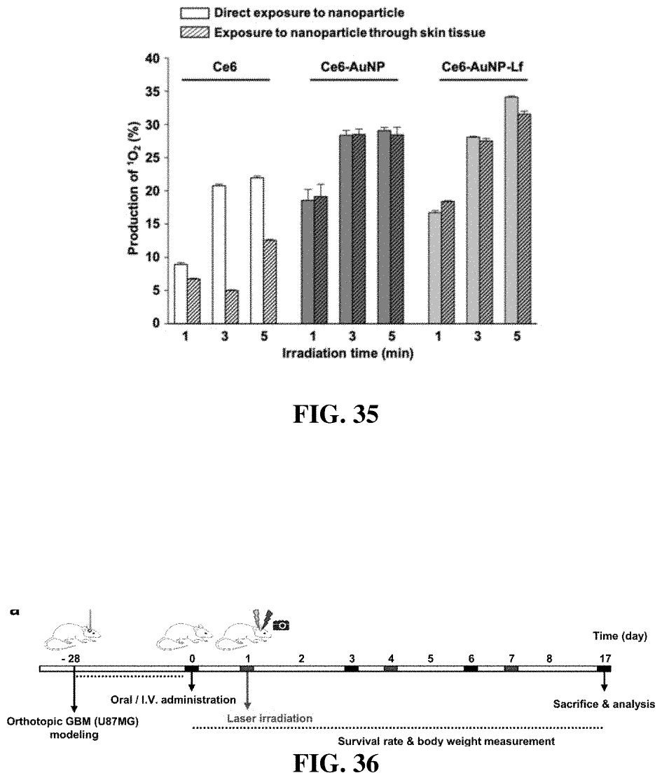

FIG. 35 is a graph showing the ROS production results of Ce6-AuNP and Ce6-AuNP-Lf in 1 cm thick live mouse skin tissue irradiated with a PDT laser (671 nm).

Referring to FIG. 35, it could be confirmed that the ROS generation of Ce6-AuNP and Ce6-AuNP-Lf was similar to that when irradiated directly without tissue intervention, and the PDT laser energy penetrating through the thick skin tissue is sufficient to generate ROS in Ce6-AuNP and Ce6-AuNP-Lf due to the MERos advantage arising from the nanoparticle complex for oral administration according to an exemplary embodiment.

### Example 8. Examination of Brain Tumor Therapeutic Effect of Nanoparticle Complex for Oral Administration Using Orthotopic GBM Mouse Model

8-1. Preparation of Orthotopic GMB Mouse Model

An orthotopic GBM mouse model for glioblastoma multiforme (GBM), which is a brain tumor tissue, was prepared as follows. Specifically, GBM cells (U87MG cells) were intracranially injected into seven-week-old male nude mice. That is, male nude mice were anesthetized with isoflurane (3%) and fixed by ear bars in a stereotaxic instrument (Stoelting Co., Ill., USA). Once each mouse was anesthetized, the scalp at the surgical position was removed and a small hole located at 2 mm right lateral and 2 mm posterior to bregma was drilled under sterile conditions. Thereafter, PBS (containing 1×10⁶U87MG cells, 8 μL) was loaded into a 26-G Hamilton syringe (Hamilton Company, Nev., USA), and then the syringe was placed on the stereotaxic apparatus. Next, after the needle of the syringe was positioned at a depth of 3 mm, cells were injected at an injection rate of 1 μL/min, followed by a waiting time of 3 minutes to prevent overflow. Next, after injection, the hole was sealed with bone wax and the scalp was closed by suturing. After this procedure, the mice were maintained for 3 weeks until the injected cells reached an appropriate size of GBM tissue. Since previous histology studies have verified that GBM exhibits a spherical shape with a diameter of about 2 mm at the site of cell injection, the boundary between GBM and normal brain tissue was established from these shapes. To evaluate GBM targeting efficacy, Ce6-AuNP and Ce6-AuNP-Lf were administered by oral (gold equivalent concentration of 0.07 μM) and IV (gold equivalent concentration of 0.012 μM) methods, respectively. 24 hours after administration, GBM and normal brain tissues were excised according to the boundary criterion and analyzed by ICP-MS and TEM. To evaluate the phototherapeutic efficacy of Ce6-AuNP-Lf in an orthotopic GBM mice model, the groups were randomized into nine groups (n=5): Con, oral No laser, IV No laser, oral PDT+PTT, oral PTT, oral PDT, IV PDT+PTT, IV PTT, and IV PDT. Then, Ce6-AuNP-Lf was administered by oral (gold equivalent concentration of 0.07 μM) and IV (gold equivalent concentration of 0.012 M) methods, respectively. 24 hours after Ce6-AuNP-Lf administration, GBM was irradiated with a single laser (PTT or PDT, 10 minutes) or a combination of lasers (PDT+PTT, for 5 minutes each). The survival rate and body weight were monitored from day 1 to day 17 after the beginning of treatment. On day 17, the brain in the mice was excised for histology analysis.

8-2. Histological Examination in Orthotopic GMB Mouse Model

Meanwhile, in vivo histological examination of the orthotopic GBM (U87MG) mouse model was performed as follows.

Specifically, tissues were immobilized in 4% paraformaldehyde for 2 days and then placed in a Leica TP1020 Semi-enclosed Benchtop Tissue Processor (Wetzlar, Germany) for washing, dehydration, clearing and paraffin infiltration of the tissue samples, followed by embedding in paraffin blocks. The paraffin blocks were cut into 6 m-thick cross sections using a Leica RM2145 Microtome (Wetzlar, Germany).

For Nissl staining, the brain slides were stained with a staining solution prepared by dissolving cresyl violet-acetate (0.2 g) in distilled water (150 mL) and a buffer solution containing acetic acid (0.1 M) and sodium acetate (0.1 M). H&E staining and TUNEL assay were used to detect necrosis and apoptosis, respectively, in the GBM regions according to the manufacturer's instruction.

Meanwhile, 24 hours after Ce6-AuNP-Lf administration, the GBM mouse model was irradiated with a single laser (PTT or PDT, 10 minutes) or a combination of lasers (PDT+PTT, for 5 minutes each). Next, on the day after laser irradiation, blood was collected from the lateral saphenous vein using a 23-gauge needle. The collected blood was allowed to clot for 30 min at room temperature and then centrifuged at 2,200×g for 10 minutes to obtain serum. Next, the obtained serum samples were divided into aliquots (1.0 mL) in polypropylene tubes and stored at −80° C. for further experiments.

8-3. Examination of Brain Tumor Therapeutic Effect in Orthotopic GMB Mouse Model

In the present example, in vivo GBM targeting of the nanoparticle complex for oral administration was evaluated using an orthotopic GBM (U87MG) mouse model, which is a brain tumor animal model, and then the therapeutic photothermal effect (PTT) and photodynamic effect (PDT) of Ce6-AuNP-Lf accumulated in GBM were evaluated.

FIG. 36 is a schematic view of a treatment cycle including drug administration, laser irradiation, survival rate and body weight change monitoring in an orthotopic GBM mouse model.

Referring to this drawing, laser irradiation was applied to the GBM-induced region of the orthotopic GBM mouse model 24 hours after administration, and the treatment cycle including Ce6-AuNP-Lf administration and laser irradiation was repeated three times. Next, the survival rates and weight changes were measured until sacrifice.

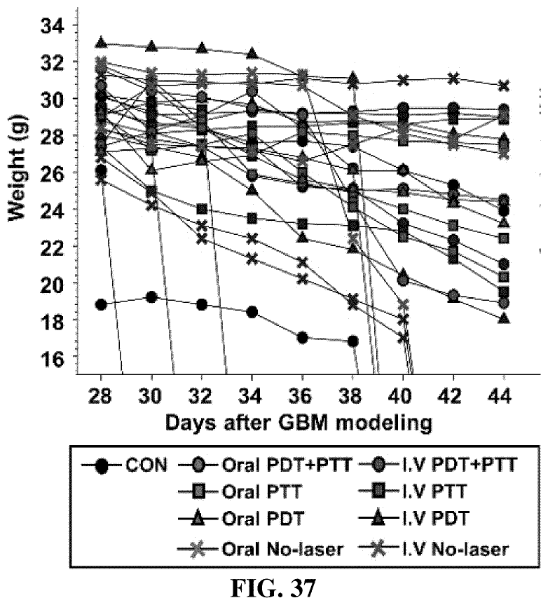

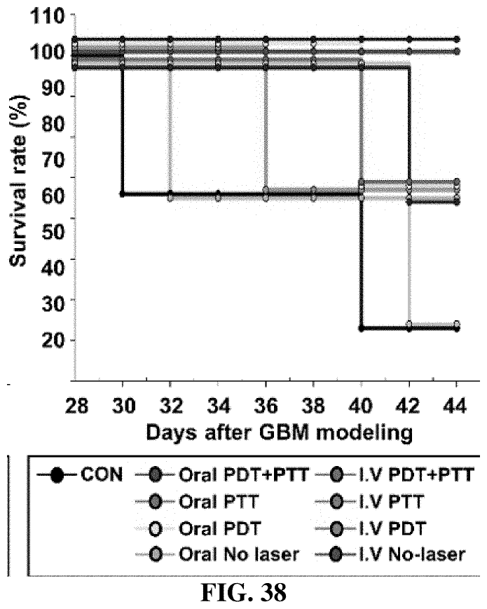

FIG. 37 is a graph showing the body weight conversion of an orthotopic GBM mouse model, and FIG. 38 is a graph showing the survival rate after administration of Ce6-AuNP-Lf up to 44 days in an orthotopic GBM mouse model.

Referring to FIGS. 37 and 38, it could be confirmed that the group in which PDT+PTT was applied after oral administration or intravenous injection maintained the body weight from the beginning of treatment (FIG. 37) and achieved a 100% survival rate until the last day (FIG. 38). Through this, it could be confirmed that weight loss is one of the parameters of repeated-administration toxicology, and that repeated administration of Ce6-AuNP-Lf itself does not have systemic toxicity.

Furthermore, in order to histologically evaluate the photothermal effect (PTT) and photodynamic effect (PDT) of the orthotopic GBM mouse model, all of the mouse models were sacrificed after the treatment.

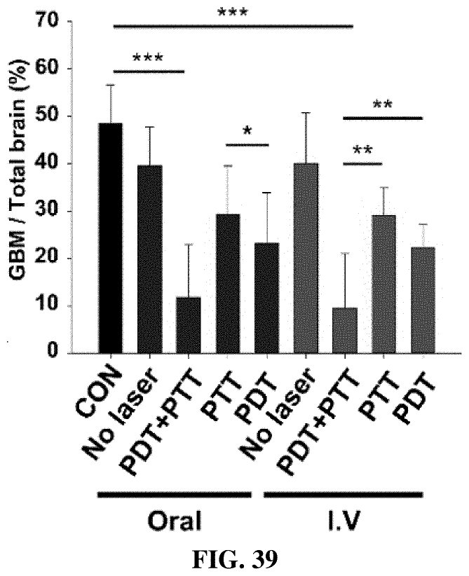

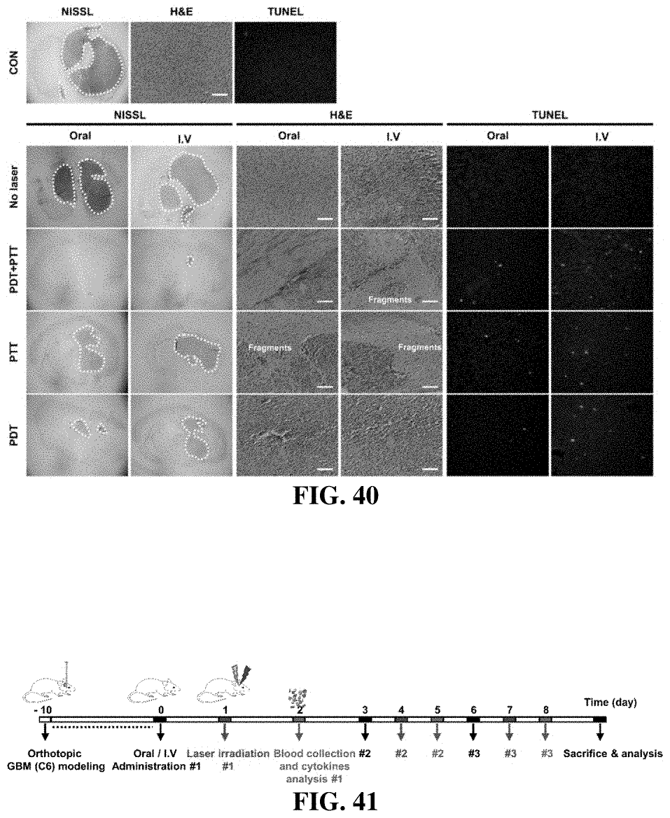

FIG. 39 is a graph showing the percentage of GBM in an orthotopic GBM mouse model. Further, FIG. 40 illustrates a set of images showing the results of Nissl staining, H&E staining, and TUNEL staining of the brain of an orthotopic GBM mouse model.

Referring to FIGS. 39 and 40, it could be confirmed that the PDT+PTT group reduced the GBM percentage to 11.8±9.8% and 9.5±10.3% in oral administration and intravenous injection groups, respectively, whereas, the No-laser, PTT and PDT groups decreased to 39.5±7.3%, 29.2±9.2% and 23.1±9.5%, respectively, in the oral administration group. In addition, it could be confirmed that in IV administration, the GBM percentage decreased to 39.9±9.6%, 29.1±5.2% and 22.2±4.7%, respectively.

Through this, it could be confirmed that the PDT+PTT combination treatment, which is the most potent treatment strategy by MERos of Ce6-AuNP, is also consistent in the orthotopic GBM mouse model.

Furthermore, the therapeutic effect of Ce6-AuNP-Lf with PDT+PTT combination treatment was evaluated by immunohistologically analyzing the extracted brain tissues.

Referring to FIG. 40, it could be confirmed that the no-laser group and control exhibited 80 to 100% intact tumor cells in H&E results. It could be confirmed that tumor cells have a dense structure and consist of polymorphic cells having a diameter of 10 to 20 m and oval nuclei. However, in the groups irradiated with the laser, it could be confirmed that the percentage of intact tumor cells decreased, and various types of cell apoptosis were observed. Meanwhile, it could be confirmed that in the groups including PTT laser irradiation, since fragments were separated from solid tumors, it can be inferred that the PTT laser irradiation causes apoptosis by physically damaging the tumor cells via Ce6-AuNP-Lf hyperthermia therapy. Further, it could be confirmed that more TUNEL-positive apoptotic cells were found in the PTT group than in the PDT group. Therefore, it could be confirmed that the ROS generated from Ce6-AuNP-Lf by PDT laser irradiation causes cancer cell apoptosis. In addition, from the results of H&E and TUNEL staining after PDT+PTT combination treatment, since it could be confirmed that there was no damage to normal brain tissue, it could be confirmed that 4 W/cm² intensity laser does not affect normal brain tissue.

### Example 9. Examination of Brain Tumor Therapeutic Effect of Nanoparticle Complex for Oral Administration Using Orthotopic GBM Rat Model

9-1. Preparation of Orthotopic GMB Rat Model

An orthotopic GBM rat model for glioblastoma multiforme (GBM), which is a brain tumor tissue, was developed from seven-week-old male Sprague Dawley rats. The rats were anesthetized using 5% isoflurane in 70% N₂O and 30% O₂, and a 2-mm hole was made in the skull. In this case, the injection point was 2.0 mm lateral to bregma and was carefully drilled using a saline (0.89% NaCl) drip (coordinates to bregma: anteroposterior, 0 mm; lateral, 2.0 mm; ventral, 4.0 mm). Then, C6 cells (1×10⁵ cells/10 μL) were injected into the cerebral cortex using a 26-gauge Hamilton syringe. Next, ten days after tumor implantation, the rats were randomly divided into 9 groups (n=5) [Con, oral No laser, intravenous (IV) No laser, oral PDT+PTT, oral PTT, oral PDT, intravenous (IV) PDT+PTT, intravenous (IV) PTT, and intravenous (IV) PDT]. In this case, Ce6-AuNP-Lf was administered by oral (gold equivalent concentration of 0.07 μM) and IV (gold equivalent concentration of 0.012 μM) methods, respectively.

24 hours after Ce6-AuNP-Lf administration, the GBM rat model was irradiated with a single laser (PTT or PDT, 10 minutes) or a combination of lasers (PDT+PTT, for 5 minutes each). Next, on the day after laser irradiation, blood was collected from the lateral saphenous vein using a 23-gauge needle. The collected blood was allowed to clot for 30 min at room temperature and then centrifuged at 2,200×g for 10 minutes to obtain serum. Next, the obtained serum samples were divided into aliquots (1.0 mL) in polypropylene tubes and stored at −80° C. for further experiments. In this case, ELISAs for IL6, IFN-7, and TNFα were performed with rat serum from each group according to the manufacturer's manual. After three repeated treatment cycles, the rats were sacrificed by perfusion and the brains were harvested and fixed with 4% paraformaldehyde for further analysis.

9-2. Histological Examination in Orthotopic GMB Rat Model

Meanwhile, in vivo histological examination of the orthotopic GBM (U87MG) rat model was performed as follows.

Specifically, tissues were immobilized in 4% paraformaldehyde for 2 days and then placed in a Leica TP1020 Semi-enclosed Benchtop Tissue Processor (Wetzlar, Germany) for washing, dehydration, clearing and paraffin infiltration of the tissue samples, followed by embedding in paraffin blocks. The paraffin blocks were cut into 6 m-thick cross sections using a Leica RM2145 Microtome (Wetzlar, Germany).

For Nissl staining, the brain slides were stained with a staining solution prepared by dissolving cresyl violet-acetate (0.2 g) in distilled water (150 mL) and a buffer solution containing acetic acid (0.1 M) and sodium acetate (0.1 M). H&E staining and TUNEL assay were used to detect necrosis and apoptosis, respectively, in the GBM regions according to the manufacturer's instruction.

Meanwhile, 24 hours after Ce6-AuNP-Lf administration, the GBM rat model was irradiated with a single laser (PTT or PDT, 10 minutes) or a combination of lasers (PDT+PTT, for 5 minutes each). Next, on the day after laser irradiation, blood was collected from the lateral saphenous vein using a 23-gauge needle. The collected blood was allowed to clot for 30 min at room temperature and then centrifuged at 2,200×g for 10 minutes to obtain serum. Next, the obtained serum samples were divided into aliquots (1.0 mL) in polypropylene tubes and stored at −80° C. for further experiments. Meanwhile, ELISAs for IL6, IFN-7, and TNFα were performed with rat serum from each group according to the manufacturer's manual. After three repeated treatment cycles, the rats were sacrificed by perfusion and the brains were harvested and fixed with 4% paraformaldehyde for further analysis. Furthermore, for immunofluorescence (IF) staining, GBM tissue slides were stained with an anti-Ki67 antibody and an anti-HMGB1 antibody and, diluted 1:100 in a mixture of phosphate buffered saline with Tween-20 (PBST) and goat serum. Next, goat anti-rabbit IgG-H&L Alexa Fluor 488 and goat anti rabbit IgG H&L Alexa Fluor 647 were used as secondary antibodies, followed by DAPI mounting.

9-3. Examination of Immune Response of Orthotopic GMB Rat Model

In the present example, in vivo GBM targeting of the nanoparticle complex for oral administration was evaluated using an orthotopic GBM (U87MG) rat model, which is a brain tumor animal model, and then the therapeutic photothermal effect (PTT) and photodynamic effect (PDT) of Ce6-AuNP-Lf accumulated in GBM were evaluated.

FIG. 41 is a schematic view including drug administration, laser irradiation, and blood collection for cytokine analysis in an orthotopic GBM (U87MG) rat model.

Referring to FIG. 41, particularly, since rats have a system that can be activated by PDT+PTT unlike nude mice, which lack an immune system, a therapeutic strategy was established through the treatment cycle as in FIG. 41 in order to confirm an immune response released by serum.

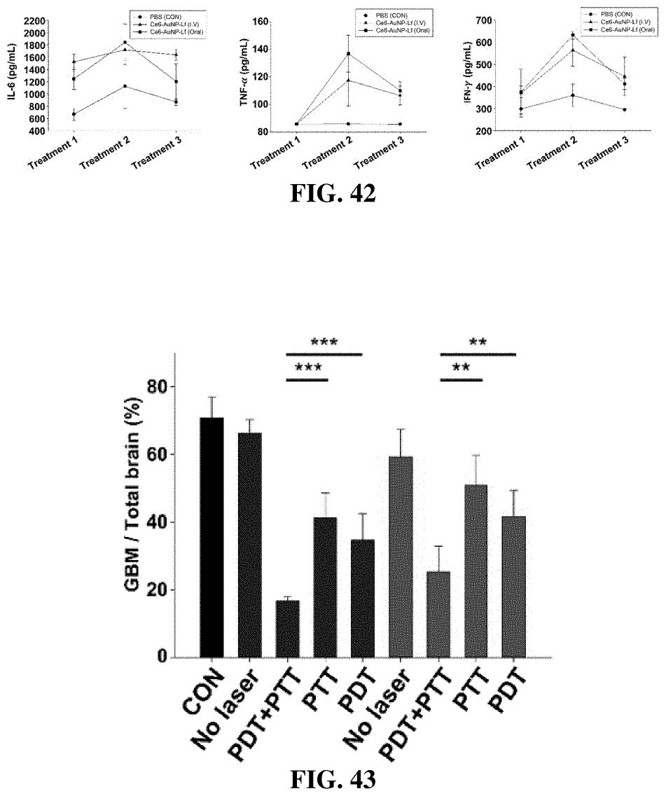

FIG. 42 is a set of graphs showing the ELISA results of pro-inflammatory cytokines IL-6, TNF-α, and TNF-γ in blood collected after Treatment #1, Treatment #2, and Treatment #3 according to FIG. 41.

Referring to FIG. 42, it could be confirmed that after treatment #1, the IL-6 was increased in the peripheral blood in both IV and oral administration groups, but IFN-γ and TNFα did not increase. Further, it could be confirmed that after treatment #2, the levels of all three cytokines (IL-6, IFN-γ, and TNFα) increased exponentially, and it could be confirmed that these cytokines were present at the highest concentration after treatment #2 and then decreased in treatment #3 but maintained higher levels than in the CON group.

That is, the phototherapy (PDT and PTT)-induced cell apoptosis generated a strong and acute local inflammatory response at treated sites to attack tumor cells, and it could be confirmed that this immune system involves the expression of the NF-κB transcription factor which induces the release of cytokines. Therefore, it can be inferred that pro-inflammatory cytokines (IL-6, IFN-γ, and TNFα) during the treatment cycle are upregulated because PDT+PTT of Ce6-AuNP-Lf subsequently destroys GBM due to an immune response.

Furthermore, the therapeutic effect of Ce6-AuNP-Lf with PDT+PTT combination treatment was evaluated by immunohistologically analyzing the extracted brain tissues.

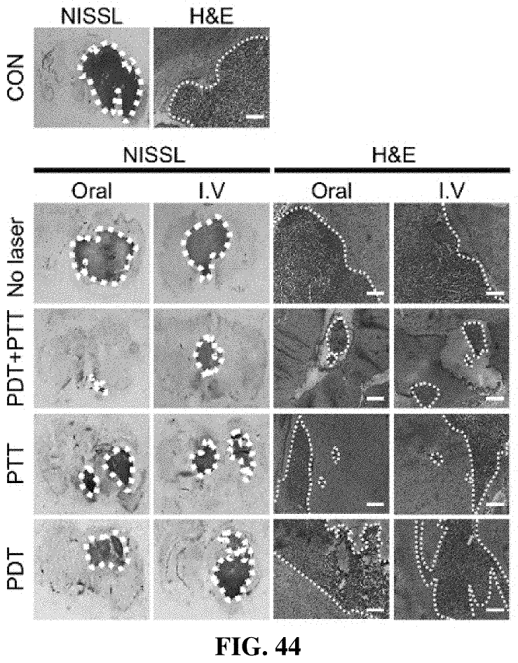

FIG. 43 is a graph showing the percentage of GBM in an orthotopic GBM rat model. Further, FIG. 44 illustrates a set of images showing the results of Nissl staining and H&E staining of the brain of an orthotopic GBM rat model.

Referring to FIGS. 43 and 44, it could be confirmed that GBM decreased to 16.7±1.1% and 25.2±6.9% in oral and IV administration groups, respectively. In addition, through Nissl staining, it could be confirmed that PDT+PTT irradiation induced cell apoptosis and necrosis in GBM, leading to the greatest amount of tumor destruction. Furthermore, consistent with the results of a GBM mice model through H&E staining, it could be confirmed that hemorrhaging and thrombosis were observed in the groups exposed to PDT laser irradiation.

That is, as a result of destruction of tumor blood vessels by ROS due to PDT, as the PDT-containing group showed a significant reduction in tumor volume, since ROS generated by PDT causes irreversible damage in endothelial cells and the vascular membrane and tumor growth is related to vasculature function due to oxygen and nutrient supply, it could be confirmed that microvasculature destruction by PDT damages tumor blood vessels, causes hemorrhaging, and destroy tumors.

Further, Ki67, tumor cell proliferation and angiogenesis markers were examined from the GBM tissue of the orthotopic GBM rat model.

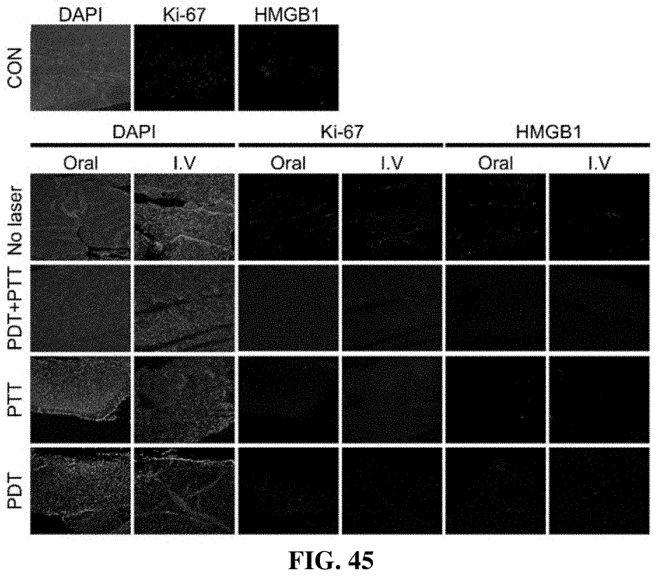

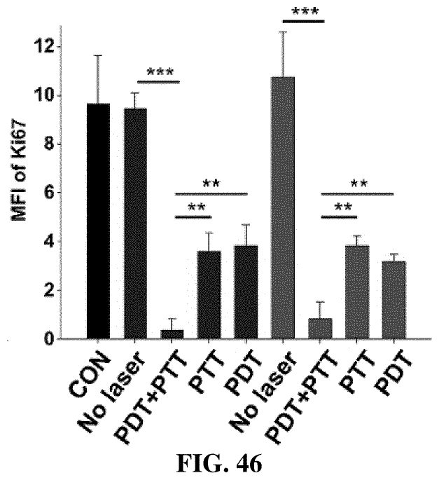

FIG. 45 illustrates a set of images showing immunofluorescent staining of Ki67 and HMGB1 in GBM tissue from an orthotopic GBM rat model. In addition, FIG. 46 is a graph quantifying the mean fluorescence intensity (MFI) of Ki67 in GBM tissue from an orthotopic GBM rat model.

Referring to FIGS. 45 and 46, it could be confirmed that Ki-67 is expressed at a high frequency in end-stage brain tumors, and the Con and No laser groups in which a large amount of GBM was detected by Nissl and H&E staining also showed high levels of Ki67 with mean fluorescence intensities (MFIs) of 9.7±0.5 and 9.5±0.5, respectively. However, since it could be confirmed that in the PDT+PTT groups, the expression of Ki67 was remarkably reduced to 0.3±0.4 and 0.8±0.6 in both oral and IV administration groups, respectively, it could be confirmed that a synergistic effect of vascular destruction and tumor cell apoptosis mediated by PDT and PTT is shown.

In addition, HMGB1 expression in GBM tissue from an orthotopic GBM rat model was investigated. High-mobility-group box 1 (HMGB1), which is an alarmin protein released from tumor cells, is considered a DAMP in cancer therapy, and acts as an endogenous ligand for toll-like receptor (TLR)-2, TLR-4, and TLR-9. Upon receptor binding, HMGB1 induces the activation of signaling pathways and immune responses, and depending on the tissue type, HMGB1 may suppress tumor growth or promote tumorigenesis. The release of DAMP may activate the immune system by inducing the maturation of dendritic cells (DCs) that eventually migrate to the lymph nodes.

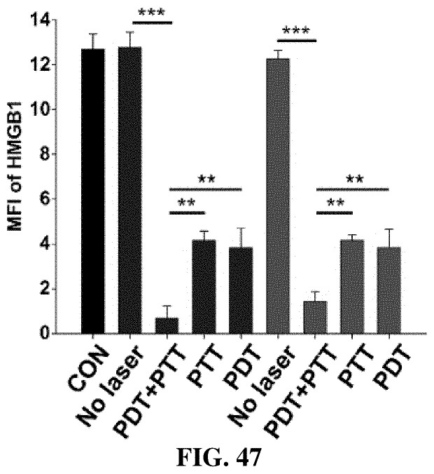

FIG. 47 is a graph quantifying the mean fluorescence intensity (MFI) of HMGB1 in GBM tissue from an orthotopic GBM rat model.

Referring to FIGS. 46 and 47, it could be confirmed that HMGB1 was expressed at high MFIs of 12.7±0.5 and 12.8±0.5 in the Con group and No laser group, respectively, which had a large amount of malignant GBM. On the other hand, it could be confirmed that the MFI gradually decreased in the PDT- or PTT-administered experimental group, and further decreased in the PDT+PTT combination administration group, and thus was shown to be 0.7±0.4 and 1.4±0.4 in the oral and IV administration groups, respectively.

In summary, with respect to the high expression of Ki67 and HMGB1 in malignant GBM, since the expression was reduced by PDT and PTT treatment, it could be confirmed that the PDT+PTT of Ce6-AuNP-Lf targeting GBM by oral administration or IV injection significantly destroy tumors.

What is claimed is:

1. A nanoparticle complex for oral administration, comprising: gold nanoparticles coated with glutathione;
a photosensitizer bonded to the gold nanoparticles; and
lactoferrin bonded to the gold nanoparticles.

2. The nanoparticle complex of claim 1, wherein the photosensitizer has a substituted thiourea group to be disulfide-bonded to the gold nanoparticles.

3. The nanoparticle complex of claim 1, wherein the photosensitizer is one or more selected from the group consisting of a chlorin-based compound, a porphyrin-based compound, a bacteriochlorin-based compound, a phthalocyanine-based compound, a naphthalocyanine-based compound, and a 5-aminoevuline ester-based compound.

4. The nanoparticle complex of claim 1, wherein the lactoferrin is surface-modified with a biocompatible polymer.

5. The nanoparticle complex of claim 4, wherein the biocompatible polymer is one or more selected from the group consisting of polyethylene glycol, polycaprolactone, polylactic acid, polyglycolic acid, polylactate-co-glycolic acid, poly(3-hydroxybutyrate-co-3-hydroxyvalerate) (PHBV), poly(L-lactide-co-caprolactone), and poly(L-lactide-co-D-lactide).

6. The nanoparticle complex of claim 1, wherein the biocompatible polymer has a substituted thiol group (—SH) to be disulfide-bonded to the gold nanoparticles.

7. The nanoparticle complex of claim 1, wherein the nanoparticle complex has an average diameter of 5 nm to 20 nm.

8. The nanoparticle complex of claim 1, wherein a weight ratio of the gold nanoparticles:the photosensitizer:the lactoferrin is 19.7 to 20.0:90.0 to 88.0:1.

9. A pharmaceutical composition for oral administration, comprising the nanoparticle complex of claim 1, an isomer thereof, or a pharmaceutically acceptable salt thereof.

10. The pharmaceutical composition of claim 9, wherein the pharmaceutical composition is for treating brain tumors.

11. A pharmaceutical composition for oral administration for photothermal therapy (PTT) or photodynamic therapy (PDT), comprising the nanoparticle complex of claim 1.

12. The pharmaceutical composition of claim 11, wherein the pharmaceutical composition is targeted to brain tumor tissue.

13. A method of treating brain tumors, the method comprising: orally administering the pharmaceutical composition of claim 11;
forming a region to be treated by accumulating the pharmaceutical composition in brain tumor tissue;
irradiating the region to be treated with output light for photodynamic therapy (PDT); and
irradiating the region to be treated with output light for photothermal therapy (PTT).
